(19) United States
(12) Patent Application Publication
(10) Pub. No.: US 2007/0129794 A1
(43) Pub. Date: Jun. 7, 2007

(54) Method and apparatus for prosthesis attachment using discrete elements

(76) Inventor: Fidel Realyvasquez, Palo Cedro, CA (US)

Correspondence Address: HELLER EHRMAN LLP, 275 MIDDLEFIELD ROAD, MENLO PARK, CA, 94025-3506, US

(21) Appl. No.: 11/538,294

(22) Filed: Oct. 3, 2006

Related U.S. Application Data
(60) Provisional application No. 60/724,074, filed on Oct. 5, 2005.

(51) Int. Cl. A61F 2/24

(52) U.S. Cl. 623/2.11; 606/200; 623/2.38; 623/1.36

(57) ABSTRACT

A percutaneous valve replacement assembly has a catheter and a plurality of discrete elements for tissue attachment. A fastener device is mounted to the catheter. The fastener device includes a working end that is movable to a first configuration to engage one of the discrete elements and a second configuration to crimp the discrete element into target tissue.

### CROSS-REFERENCE TO RELATED APPLICATIONS

This application claims the benefit of U.S. Ser. No. 60/724,074, filed Oct. 5, 2005, which application is fully incorporated herein by reference.

### BACKGROUND OF THE INVENTINO

1. Technical Field

The invention relates to apparatus and methods for prosthesis attachment and is especially useful in aortic valve repair procedures.

2. Background Art

Essential to normal heart function are four heart valves, which allow blood to pass through the four chambers of the heart in one direction. The valves have either two or three cusps, flaps, or leaflets, which comprise fibrous tissue that attaches to the walls of the heart. The cusps open when the blood flow is flowing correctly and then close to form a tight seal to prevent backflow.

The four chambers are known as the right and left atria (upper chambers) and right and left ventricles (lower chambers). The four valves that control blood flow are known as the tricuspid, mitral, pulmonary, and aortic valves. In a normally functioning heart, the tricuspid valve allows one-way flow of deoxygenated blood from the right upper chamber (right atrium) to the right lower chamber (right ventricle). When the right ventricle contracts, the pulmonary valve allows one-way blood flow from the right ventricle to the pulmonary artery, which carries the deoxygenated blood to the lungs. The mitral valve, also a one-way valve, allows oxygenated blood, which has returned to the left upper chamber (left atrium), to flow to the left lower chamber (left ventricle). When the left ventricle contracts, the oxygenated blood is pumped through the aortic valve to the aorta.

Certain heart abnormalities result from heart valve defects, such as valvular insufficiency. Valve insufficiency is a common cardiac abnormality where the valve leaflets do not completely close. This allows regurgitation (i.e., backward leakage of blood at a heart valve). Such regurgitation requires the heart to work harder as it must pump both the regular volume of blood and the blood that has regurgitated. Obviously, if this insufficiency is not corrected, the added workload can eventually result in heart failure.

Another valve defect or disease, which typically occurs in the aortic valve is stenosis or calcification. This involves calcium buildup in the valve which impedes proper valve leaflet movement.

In the case of aortic valve insufficiency or stenosis, treatment typically involves removal of the leaflets and replacement with valve prosthesis. However, known procedures have involved generally complicated approaches that can result in the patent being on cardiopulmonary bypass for an extended period of time.

There is a need for improved valvular repair apparatus and methods that use minimally invasive techniques and/or reduce time in surgery. Although known technology have described methods to replace a human aortic valve with a prosthesis, these methods are, however, designed to be used while the patient is on cardiopulmonary bypass and an open aorta technique. It is understood that there are potentially adverse effects from cardiopulmonary bypass. Recently, methods have been introduced to insert a stented aortic valve using percutaneous techniques but, unfortunately, the native aortic valve is left in situ and presently limited to very ill patients not suitable for valve replacement by conventional means. The need remains for further improved methods of valve repair and/or replacement.

### SUMMARY OF THE INVENTION

Accordingly, an object of the present invention is to provide an improved valvular repair apparatus and methods that use minimally invasive techniques and/or reduce time in surgery.

Another object of the present invention is to provide an apparatus, and method for securing a valve prosthesis to a target tissue area.

A further understanding of the nature and advantages of the invention will become apparent by reference to the remaining portions of the specification and drawings.

These and other objects of the present invention are achieved in a percutaneous valve replacement assembly that has a catheter and a plurality of discrete elements for tissue attachment. A fastener device is mounted to the catheter. The fastener device includes a working end that is movable to a first configuration to engage one of the discrete elements and a second configuration to crimp the discrete element into target tissue.

In another embodiment of the present invention, a method of percutaneous valve replacement accesses a femoral blood vessel and inserts a guidewire to guide a catheter to a target site in the heart. The catheter is advanced along the guidewire in a collapsed configuration. At least one of a plurality of discrete elements are positioned on the catheter for tissue attachment. A fastener device is mounted to the catheter. The fastener device includes a working end that is movable to a first configuration to engage one of the discrete elements and a second configuration to crimp the discrete element into target tissue.

In another embodiment of the present invention, a minimally invasive prosthesis attachment device includes a shaft and a plurality of discrete elements for tissue attachment. A clamp system holds each of the discrete elements in position to engage target tissue. A fastener device is mounted to the shaft. The fastener device includes a working end that is movable to a first configuration to engage one of the discrete elements and a second configuration to crimp the discrete element into target tissue.

### BRIEF DESCRIPTION OF THE DRAWINGS

FIG. 1 illustrates one embodiment of a device of the present invention that can be used to fasten a prosthesis to tissue using a plurality of discrete elements.

FIG. 2 illustrates one embodiment of a valve prosthesis that can be inserted onto the device of FIG. 1 and ready for crimping.

FIG. 3 illustrates one embodiment of the crimp tool that can be used with the present invention.

FIG. 4 illustrates one embodiment of a clamp system of the present invention.

FIG. 5 illustrates one embodiment of a clamp system of the present invention being extended further out from the opening of the valve prosthesis

FIG. 6 illustrates that in one embodiment of the present invention, one set of fingers are collapsed radially inward to define a smaller circumferential area, and a second set of fingers is then also collapsed radially inward.

FIG. 7 illustrates an embodiment of the present invention where the second set of fingers is in a radially compressed configuration that allows the clamp system to be removed from the patient.

FIG. 8 illustrates an embodiment of a fastener removal device of the present invention

FIG. 9 illustrates another embodiment of the present invention with a scissor type action to disengage the discrete element from the tissue and allows the prosthesis to be removed.

FIG. 10 illustrates an embodiment of the present invention that has a dual tang staple section.

FIG. 11 illustrates one embodiment of the present invention with the elements in a J-configuration with one end being curved and engaged in the valve prosthetic.

FIG. 12 illustrates one embodiment of the present invention with the elements crimped into a C-configuration,

FIG. 13 illustrates an embodiment of the present invention with the elements being unclamped from the clamp system

FIG. 14 illustrates an embodiment of the present invention with the clamp system being moved forward as indicated by the arrow.

FIG. 15 illustrates an embodiment of the present invention with the system at a reduced circumference.

FIG. 16 illustrates an embodiment of a crimping tool used with the present invention,

FIG. 17 illustrates a close-up view of an active end of the FIG. 16 crimping tool.

FIG. 18 illustrates a distal working end of the FIG. 16 crimping tool.

FIG. 19 illustrates one embodiment of a side view of the FIG. 16 crimping tool.

FIG. 20 illustrates a cross-sectional view of the FIG. 19 device.

FIG. 21 illustrates a perspective view of a distal end of a valve prosthesis with a crimping tool of the present invention to secure the elements to tissue and fasten the prosthesis in place.

FIG. 22 is an exploded perspective view of one embodiment of the device of the present invention with the crimping tool.

FIG. 23 is a close-up view of a distal end of a prosthesis using a crimping tool of the present invention which can reciprocate longitudinally to crimp discrete elements.

FIG. 24 illustrates one embodiment of the present invention for use in a percutaneous prosthesis fastening procedure.

FIG. 25 illustrates the jig from the FIG. 24 embodiment compressing the element into a C-configuration.

FIG. 26 shows the element being released from the jig of FIG. 25.

FIG. 27 shows an attachment device with the jig of FIG. 25 for shaping and delivering the elements.

FIG. 28 shows the jig compressing the element into the C-configuration.

FIG. 29 shows the first element being released.

FIG. 30 shows a rotational device coupled to a geared ring that has been turned and has indexed the device to a different location along the circumference of the valve prosthesis.

FIG. 31 shows the jig compressing the element into the C-configuration.

FIG. 32 shows the second element being released.

FIG. 33 illustrates an embodiment of the present invention with a temporary valve and a fastening device mounted about a central shaft that rotates to index the fastening device.

FIGS. 34 through 35 illustrate the crimping and releasing of the fastener from the device of FIG. 33.

FIG. 36 illustrates that the shaft from FIG. 33 can be rotated to reposition the device to deliver and/or crimp the elements in various locations.

FIGS. 37 through 38 illustrate the FIG. 33 device with crimping and releasing of the fastener at a second location.

FIG. 39 illustrates another view of the temporary valve from FIG. 33.

FIG. 40 shows the temporary valve of FIG. 33 in an open configuration with the flaps opened.

FIG. 41 is a cross-sectional view that more clearly shows a compressed Cribier-Edwards Valve positioned for deployment.

FIG. 42 illustrates the Cribier-Edwards Valve deployed in place and ready for attachment.

FIG. 43 illustrates an embodiment of the present invention with the device ready to deploy and crimp the element.

FIG. 44 shows the element 20 being crimped.

FIG. 45 shows an embodiment of the device of the present invention lowering the jig away from the element in order to release it.

FIG. 46 illustrates rotation of the shaft of the FIG. 33 device to position the device in a new location and deploy another fastener.

FIGS. 47 and 48 illustrate the crimp and release of the fastener of the FIG. 33 device.

FIGS. 49 and 50 are cross-sectional views of the prosthesis in place and attached by a plurality of discrete elements.

FIG. 51 illustrates that the shape of a crimped element can include a rise or bump along a mid-section to grip the prosthesis.

FIGS. 52 through 55 show aortic root attachment methods.

FIG. 55 illustrates coronary attachments completed and prepared for distal aortic anastomosis.

FIG. 56 illustrates a single tang staple alternative.

### DESCRIPTION OF THE SPECIFIC EMBODIMENTS

It is to be understood that both the foregoing general description and the following detailed description are exemplary and explanatory only and are not restrictive of the invention, as claimed. It may be noted that, as used in the specification and the appended claims, the singular forms “a”, “an” and “the” include plural referents unless the context clearly dictates otherwise. Thus, for example, reference to “a material” may include mixtures of materials, reference to “a chamber” may include multiple chambers, and the like. References cited herein are hereby incorporated by reference in their entirety, except to the extent that they conflict with teachings explicitly set forth in this specification.

In this specification and in the claims which follow, reference will be made to a number of terms which shall be defined to have the following meanings:

“Optional” or “optionally” means that the subsequently described circumstance may or may not occur, so that the description includes instances where the circumstance occurs and instances where it does not. For example, if a device optionally contains a feature for using an inflatable valve support, this means that the inflatable feature may or may not be present, and, thus, the description includes structures wherein a device possesses the inflatable feature and structures wherein the inflatable feature is not present.

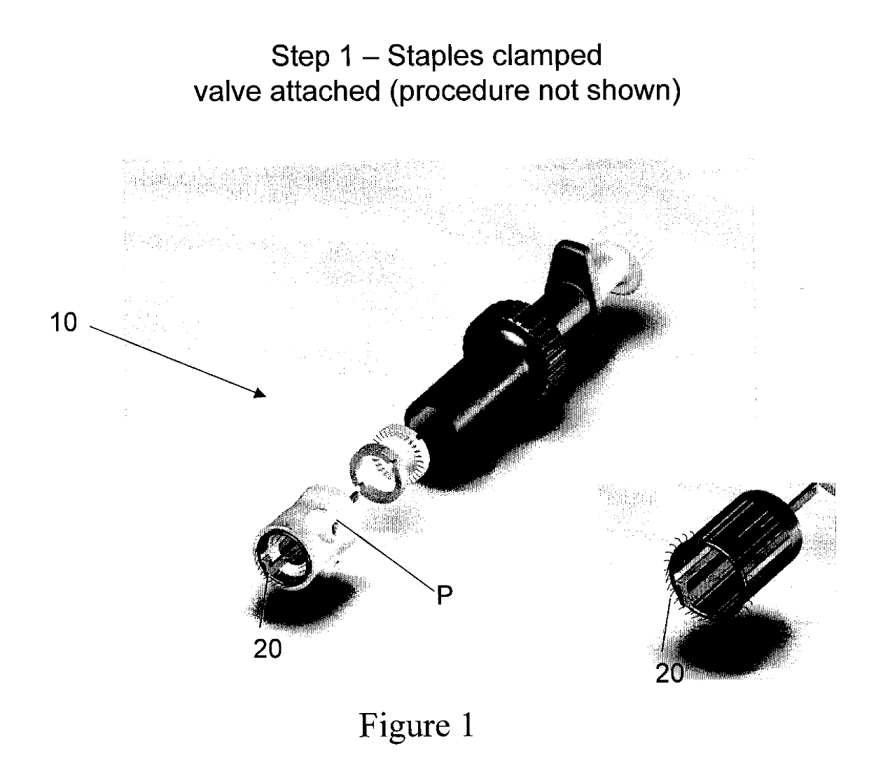

Referring now to FIG. 1, embodiments of the present invention now describe methods and improvements to fasten a prosthetic device using an open or minimally invasive approach. By nonlimiting example, the prosthetic device may be a valve prosthesis for delivery into the heart. It should be understood however, that embodiments of the device may be adapted to deliver prosthetic devices for use in other parts of the body such as the intestine, other blood vessels, or other organs using valves. The prosthetic device may be expandable between a collapsed and expanded configuration.

FIG. 1 shows one embodiment of a device 10 that can be used to fasten the prosthesis P to tissue using a plurality of discrete elements 20. In the present embodiment, the discrete elements 20 may be individual wires that are formed to be staples. The wires may be made of nitinol, stainless steel, or other suitable material.

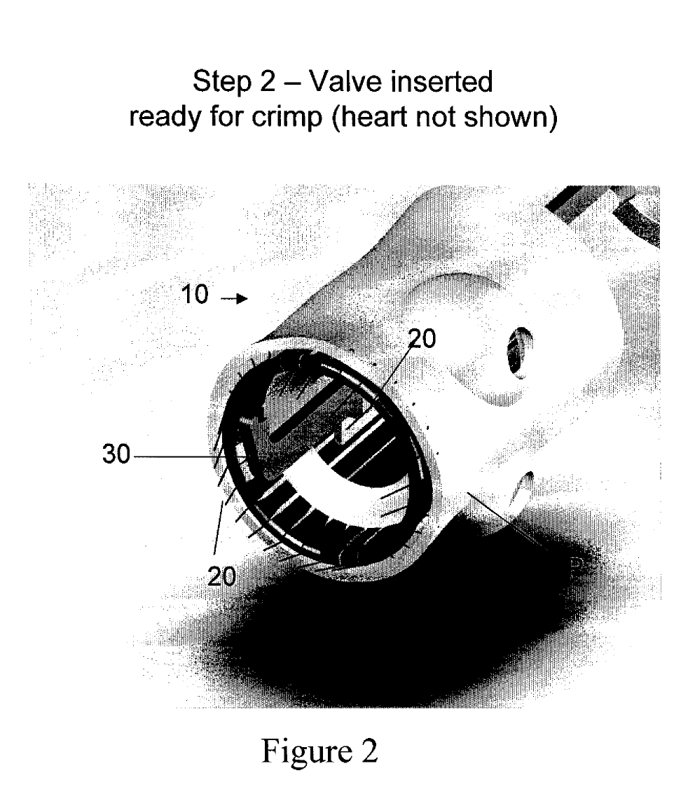

FIG. 2 shows the valve prosthesis P inserted onto device 10 and ready for crimping (heart not shown). The discrete elements 20 may have one end that is penetrating into the valve prosthesis P and another end the remains straight and will be crimped to attach the prosthesis P to tissue. FIG. 2 also shows a crimp tool 30 for use in reshaping the discrete elements 20 to attach to target tissue. By way of nonlimiting example, the crimp tool 30 may reciprocate longitudinally to crimp each discrete element 20. Some embodiments may have the tool 30 crimp more than one element 20 at a time. The tool 30 may also be rotated to engage the various elements 20 along the circumference of the valve prosthesis P.

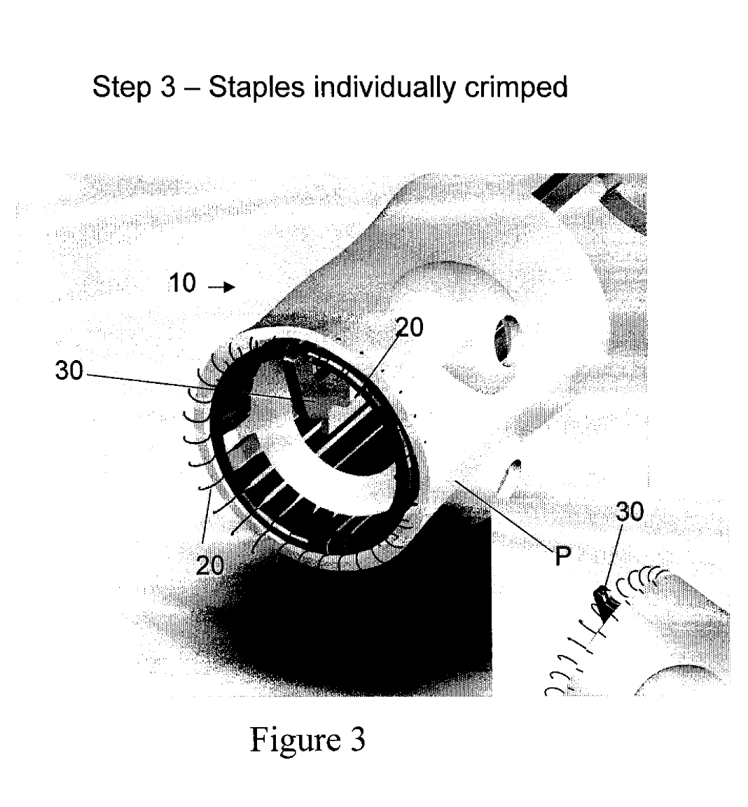

FIG. 3 shows one embodiment of the crimp tool 30 in a retracted position and crimping a discrete element 20 into shape to engage tissue (not shown). In this present embodiment, the crimp tool 30 will rotate all the way around the circumference and crimp each element 20 in place.

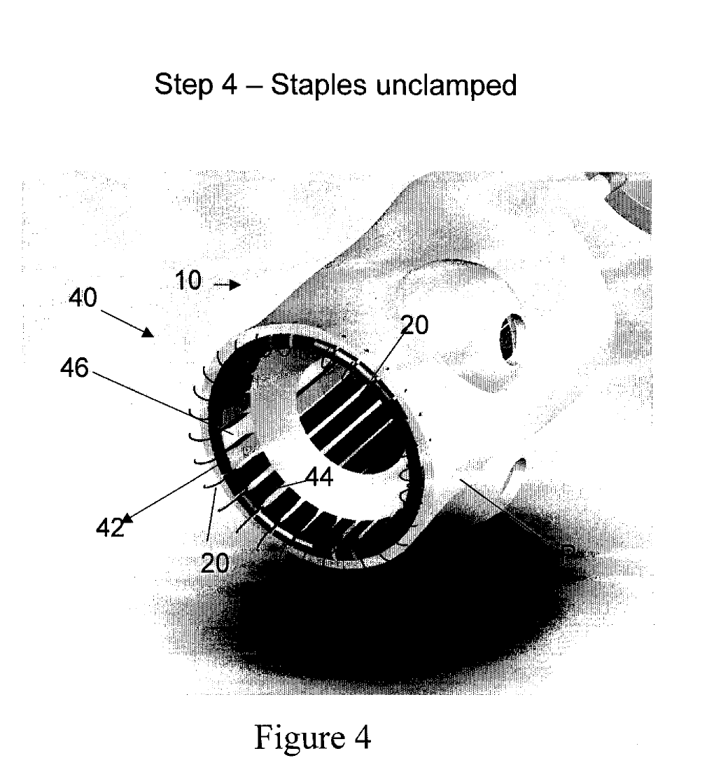

FIG. 4 shows the clamp system 40 extend forward as indicated by arrow 42 to begin to release each of the discrete elements 20. The elements 20 are at this point fastened to both the valve prosthesis P and the tissue (not shown for ease of illustration). The movement forward of the clamp system 40 increase the gap 44 between each finger 46 of the clamp system 40 and thus releases contact with the elements 20.

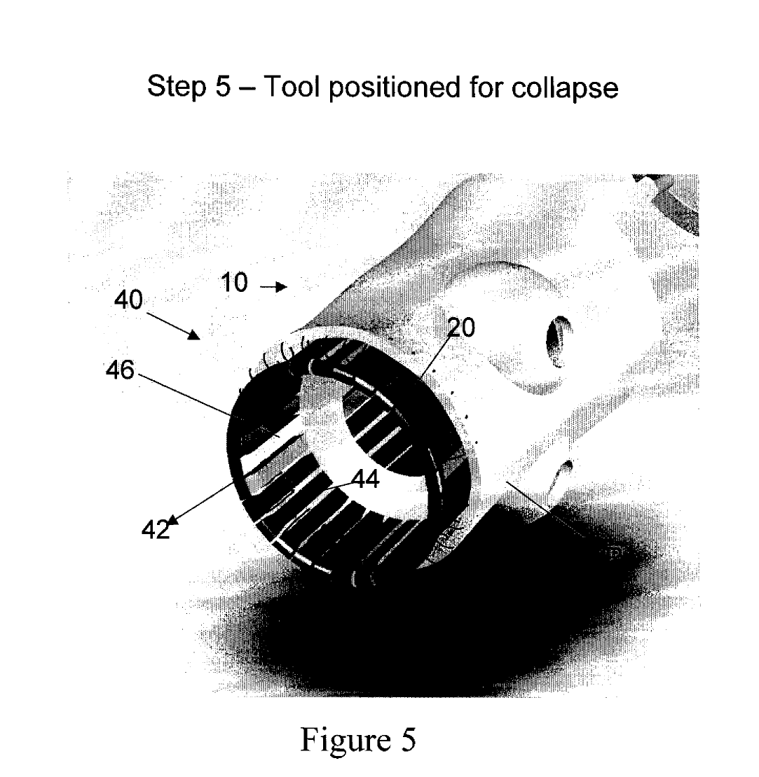

FIG. 5 shows the clamp system 40 being extended further out from the opening of the valve prosthesis P. As will be seen later, this provides sufficient room for the individual fingers to be collapsed and the entire clamp system 40 retracted away.

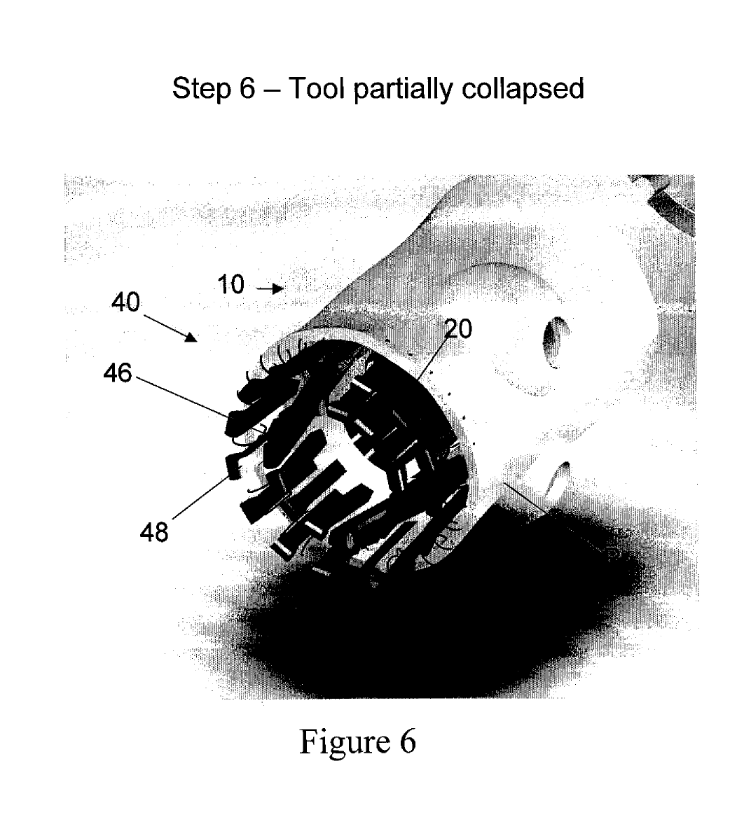

FIG. 6 shows how one set of fingers 46 are collapsed radially inward to define a smaller circumferential area. A second set of fingers 48 will then also be collapsed radially inward so that the entire circumference of the clamp system 40 is reduced to a size sufficient small to allow for extraction from the center of the valve prosthesis P.

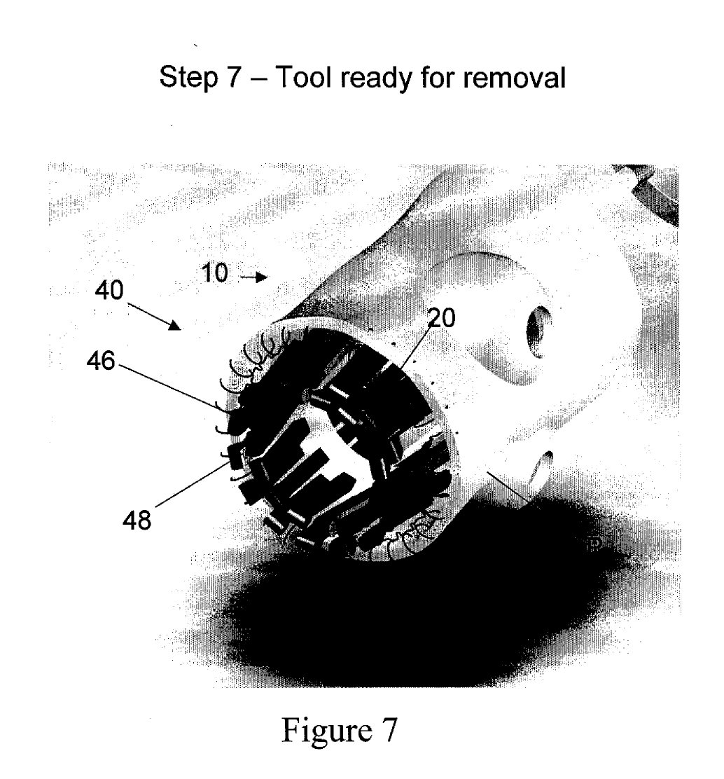

FIG. 7 shows the second set of fingers 48 in a radially compressed configuration and allowing the clamp system 40 to be removed from the patient.

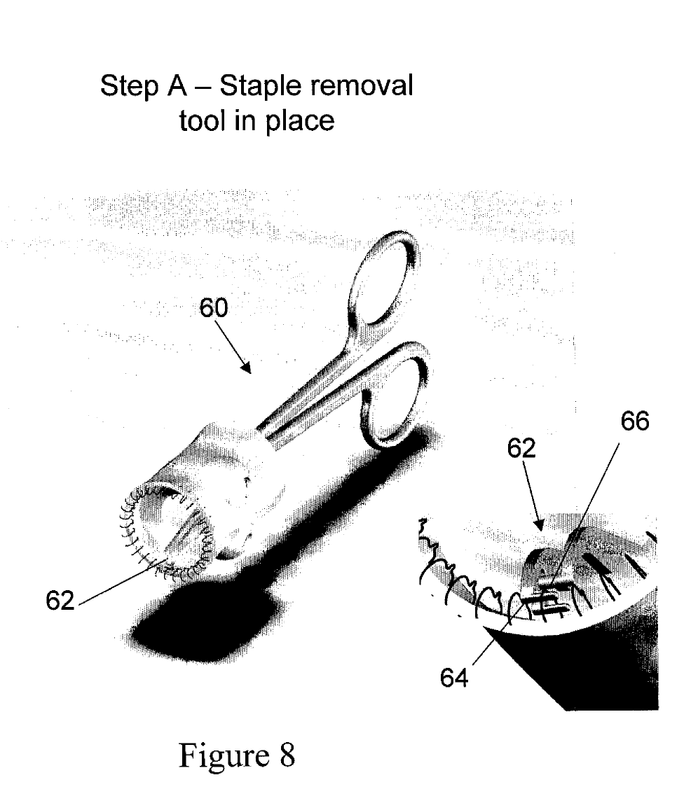

Referring now to FIG. 8, one embodiment of a fastener removal device 60 will now be described. FIG. 8 shows the distal end 62 of the removal device 60 configured to grip and remove the individual discrete elements 20. In this embodiment, one part of the distal end 62 has two prongs 64 while a second part of distal end 62 has one prong 66 the meshes between the two prongs of the other part.

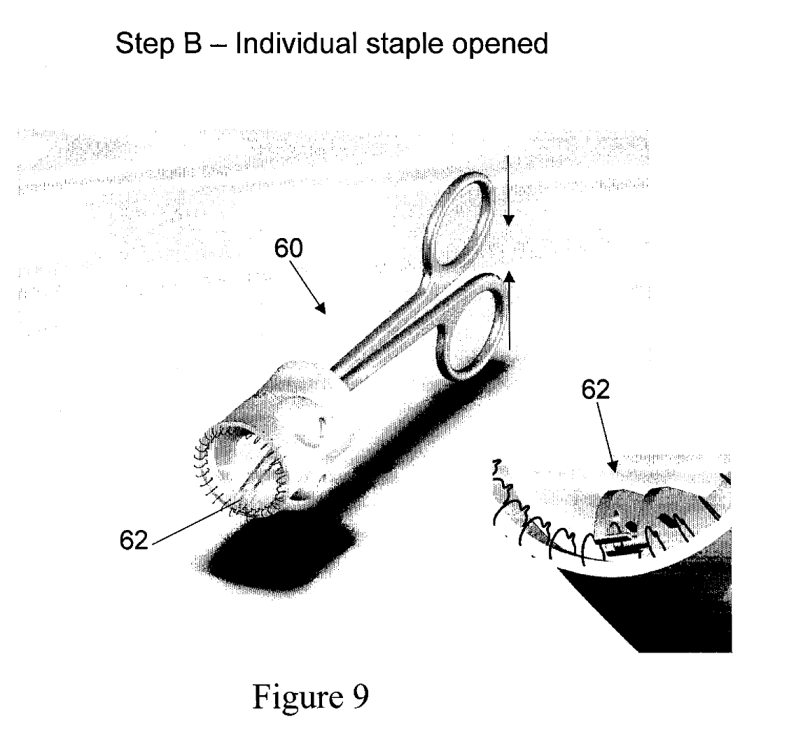

As seen in FIG. 9, as the scissor type action of the device 60 is closed, the prong 66 will move between prongs 64. This movement will bend the center of the discrete element 20 and cause the ends of the discrete element 20 to bend upward. This disengages the discrete element 20 from the tissue and allows the prosthesis P to be removed when all the elements 20 are disengaged.

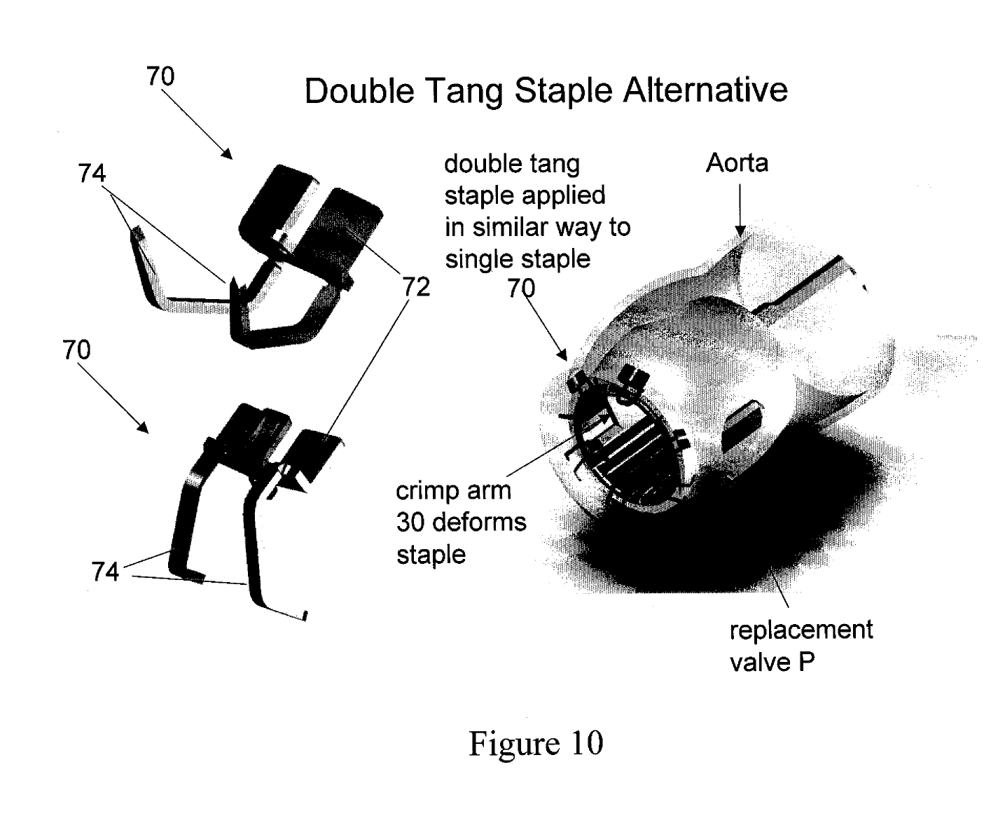

Referring now to FIG. 10, yet another embodiment of the present invention will now be described. FIG. 10 shows an embodiment using a dual tang staple section 70. Again, these sections may be held in place a clamp system 40 which may have gaps that are expandable and compressible to grip or release the dual tang staple sections 70. The sections 70 may have outer portions 72 which help to secure against the prosthesis P. The dual tangs 74 may be deformed by the crimping tool 30 to secure the device in place.

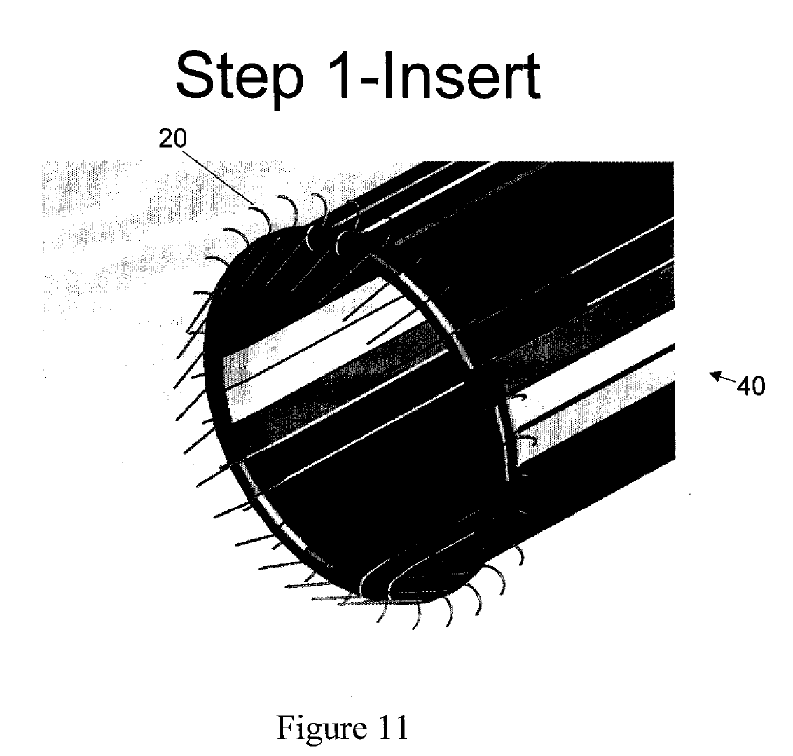

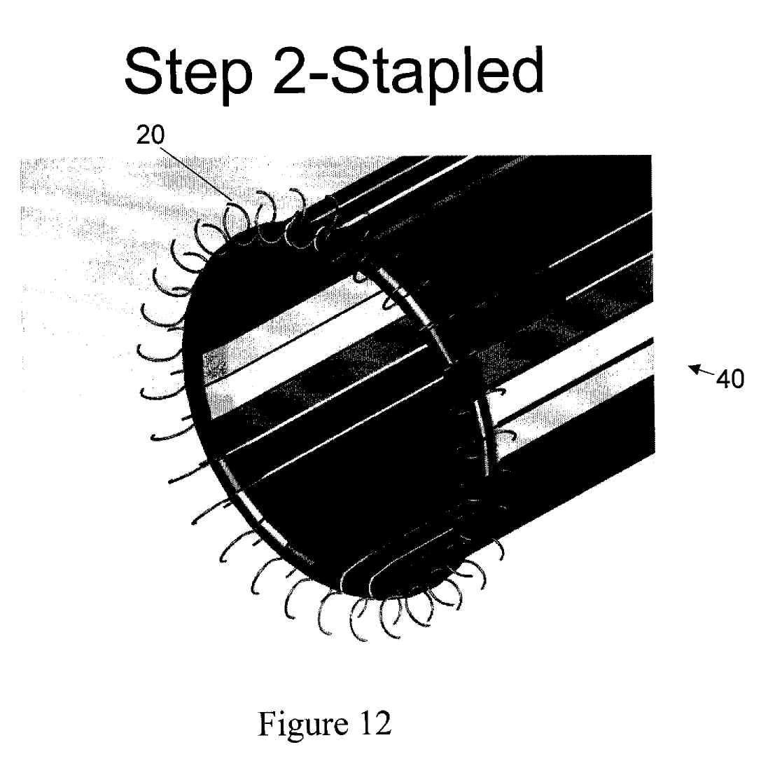

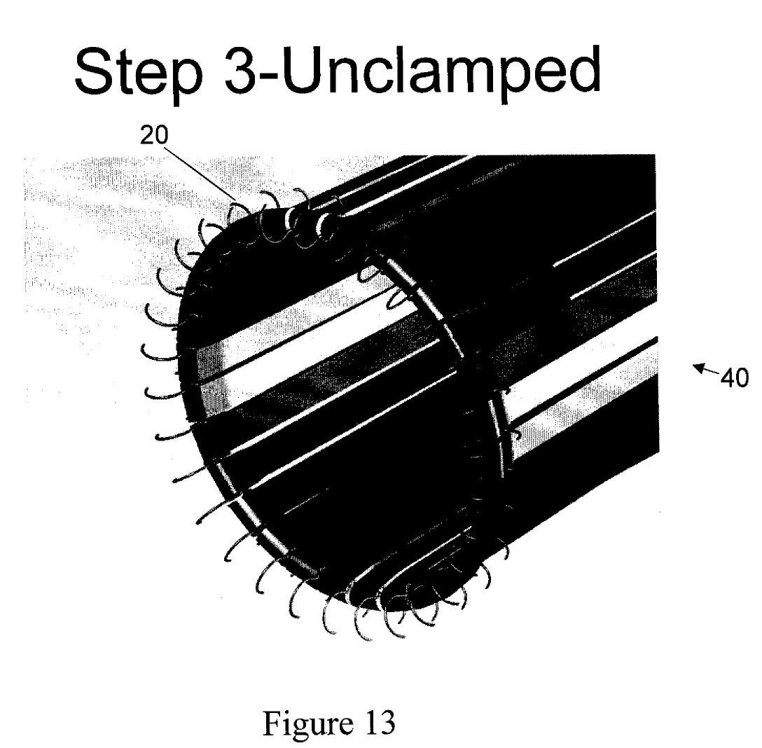

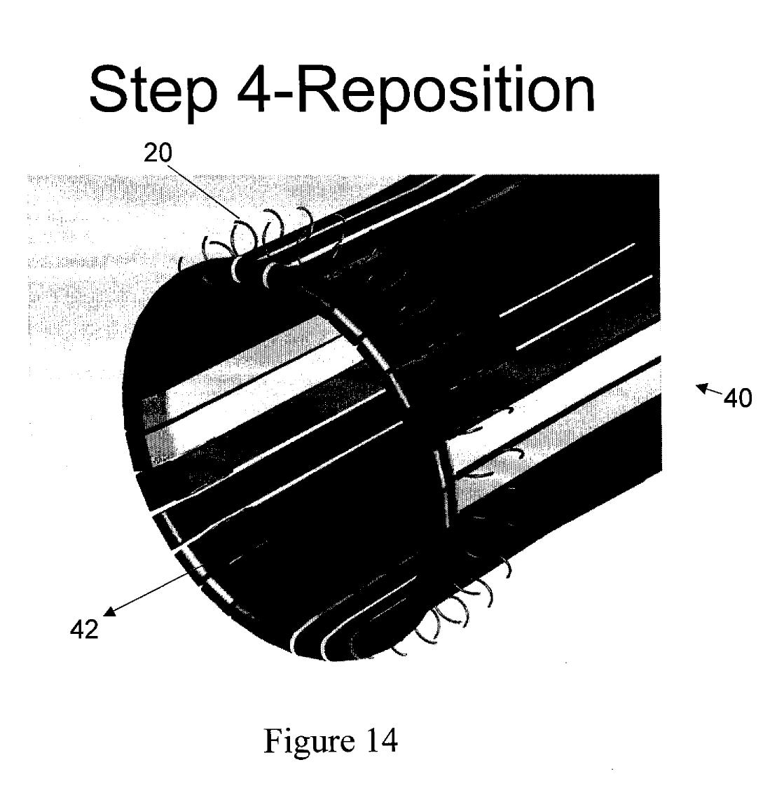

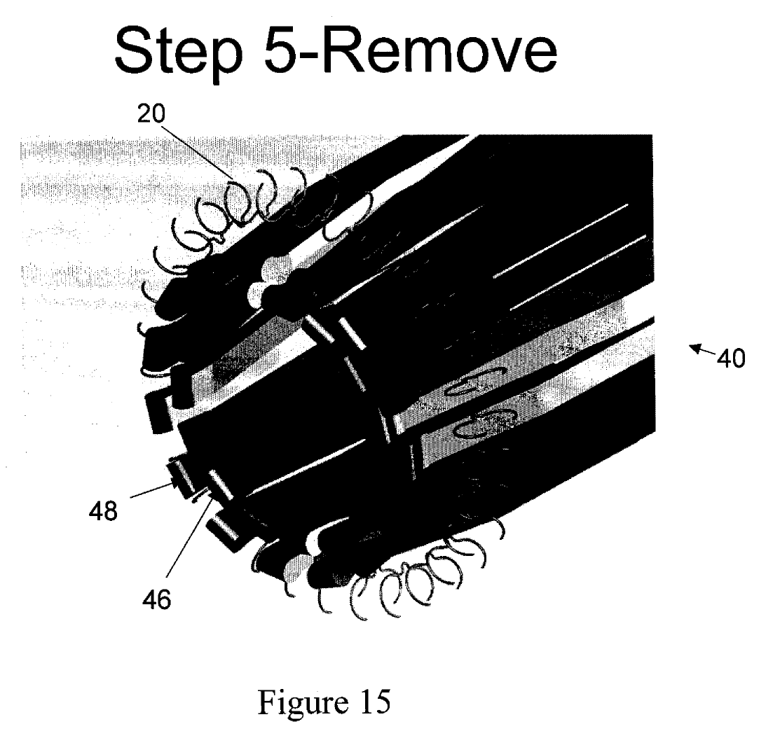

Referring now to FIGS. 11 to 15, the individual elements 20 and the clamp system 40 are shown in more detail with other elements (prosthesis, tissue, etc.) not shown for ease of illustration. FIG. 11 shows the elements 20 in a J-configuration where one end is curved and engaged in the valve prosthetic. FIG. 12 shows that the elements 20 have been crimped into a more C-configuration, where the element 20 is embedded into tissue. FIG. 13 shows the elements 20 being unclamped from the clamp system 40. In this embodiment, this shows the fingers 46 and 48 being moved radially outward. FIG. 14 shows the clamp system 40 being moved forward as indicated by arrow 42. FIG. 15 shows the system 40 at a reduced circumference.

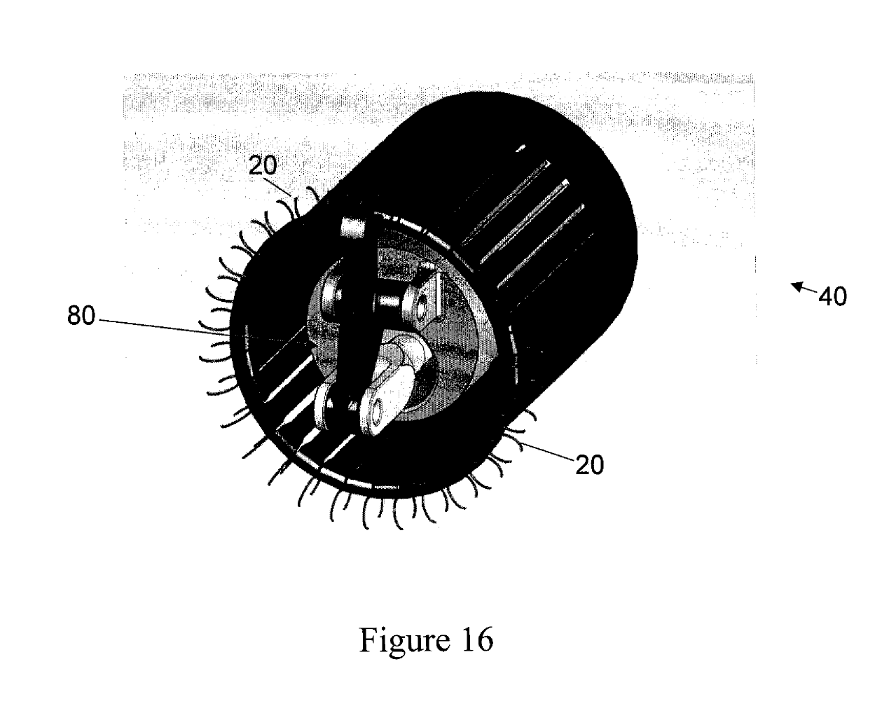

Referring now to FIG. 16, another embodiment of a crimping tool according to the present invention will now be described. The crimping tool 80 may use a four-bar linkage design using various hinges to move the active end 82 of the tool 80 to crimp the elements 20.

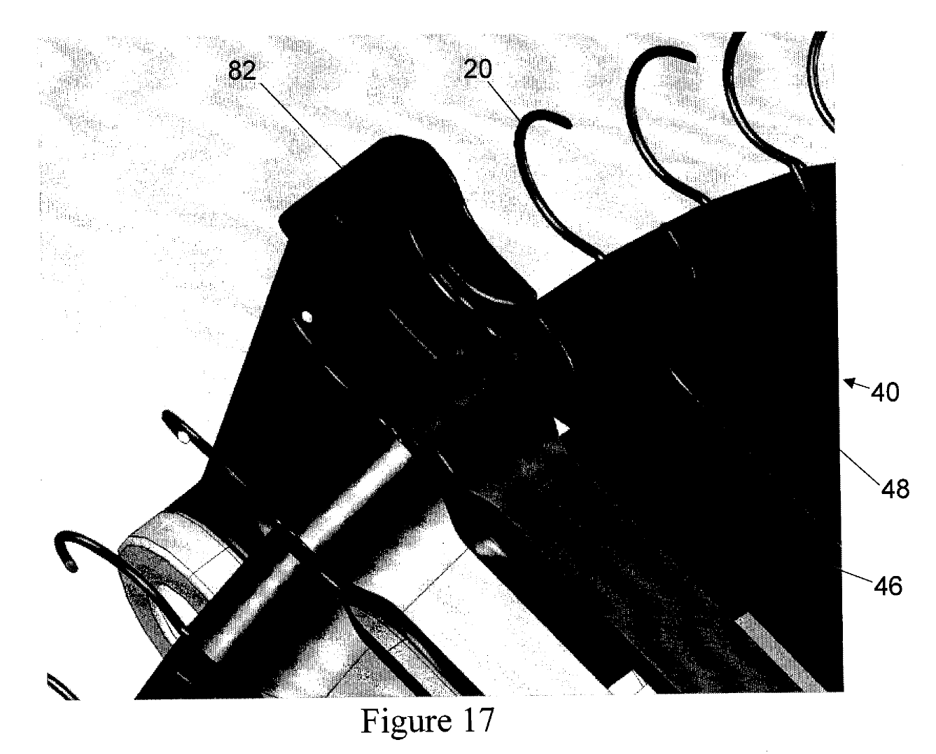

Referring now to FIG. 17, a close-up view of active end 82 is shown. The end 82 may include a groove or slot shaped to deform the element 20 in curved manner to engage tissue.

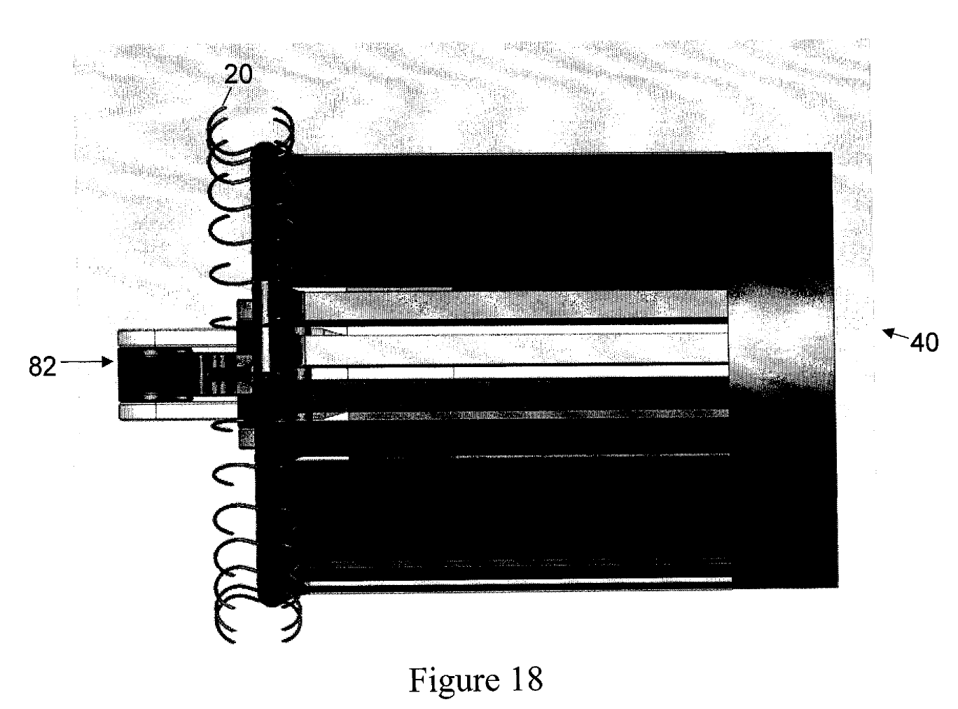

FIG. 18 shows a distal working end 82 positioned to engage one of the elements 20.

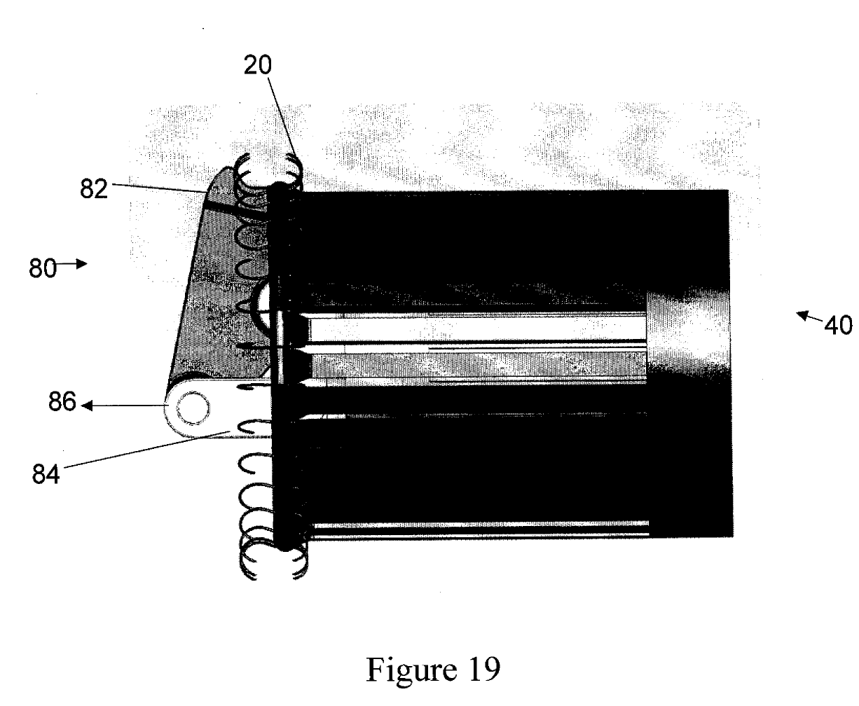

FIG. 19 shows a side view that more clearly illustrates how the crimp tool 80 works. The push bar 84 may be extended in direction 86 which causes the working end 82 of crimp tool 80 to press against the discrete element 20.

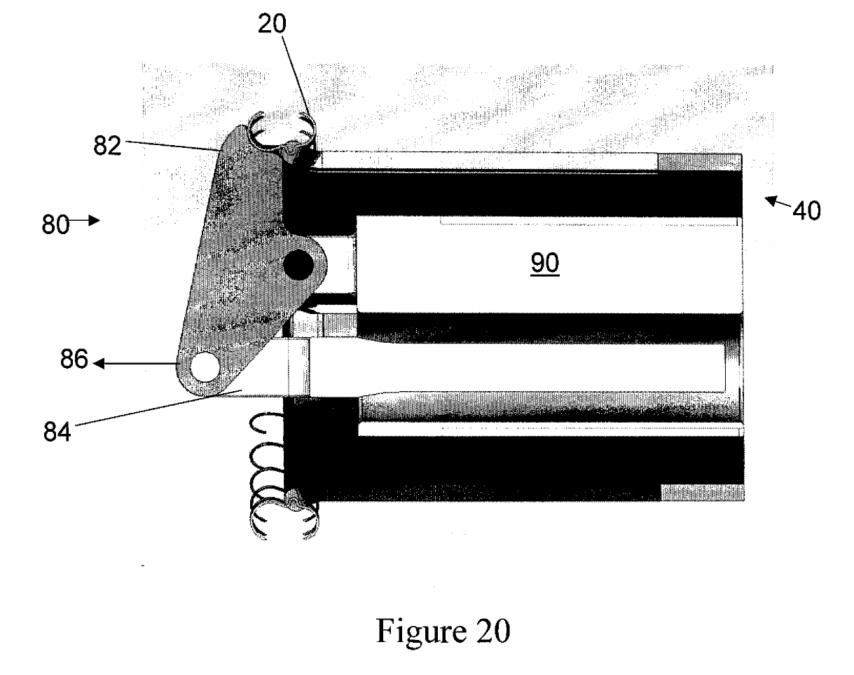

FIG. 20 shows a cross-sectional view of the device shown in FIG. 19. FIG. 20 shows that crimp tool 80 is hinged to the bar 84 and shaft 90 which provides a base against which the tool 80 can pivot.

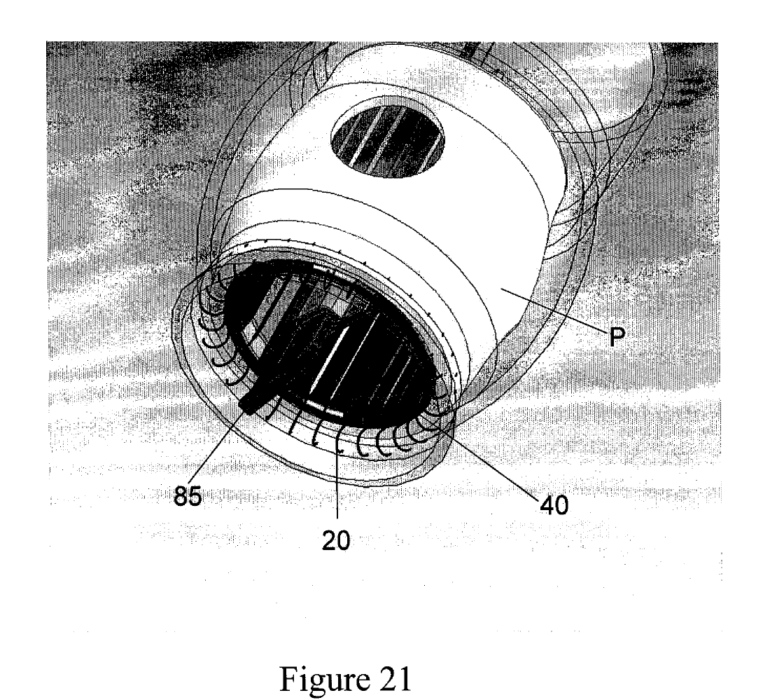

FIG. 21 shows a perspective view of a distal end of the valve prosthesis P with the crimping tool 80 used to secure the elements 20 to tissue and fasten the prosthesis P in place. The tool 80 may be indexed around the entire circumference of the valve prosthesis P to attach the elements 20 in place.

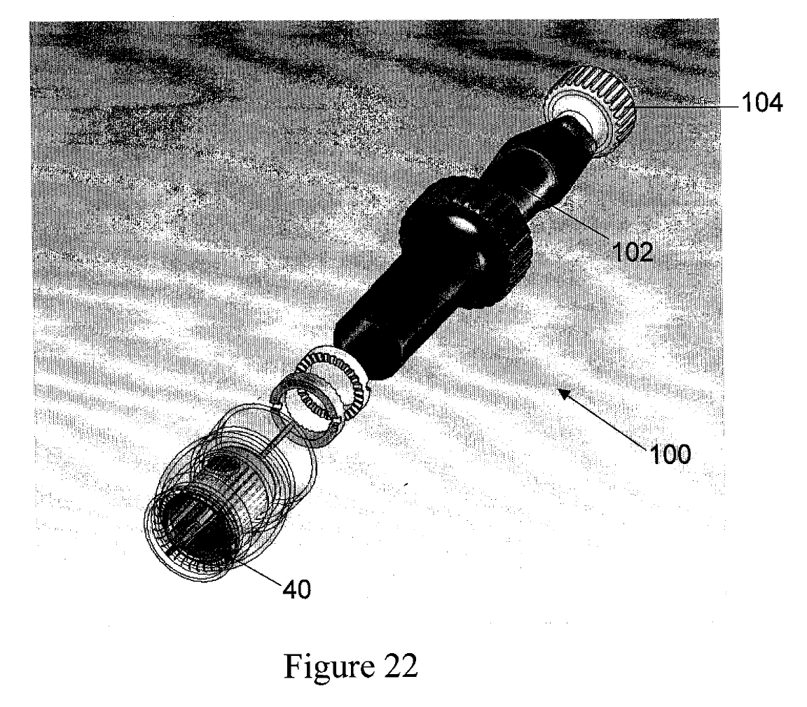

FIG. 22 shows an exploded perspective view of the device 100 having the crimping tool 80. The device 100 has a shaft 102 for actuating the crimper and a rotational device 102 for indexing the crimping tool 80 around to crimp all of the elements 20.

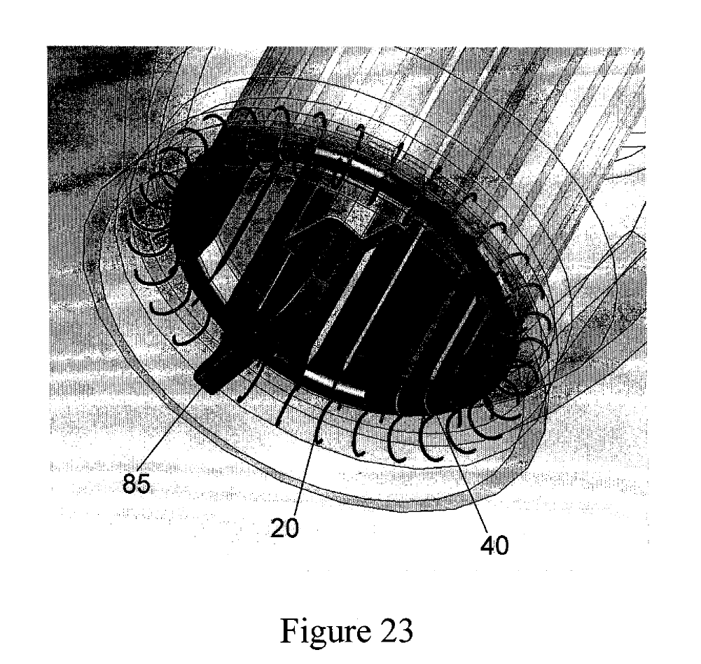

FIG. 23 is a close-up view of a distal end of prosthesis P using a crimping tool 85 which can reciprocate longitudinally to crimp discrete elements 20. The use of discrete fasteners may advantageously maintains valve compliance. The type shown in FIG. 23 provides familiar fastening and expected wider industry acceptance. The individual staples or elements 20 allows for flexible spacing and number of staples. The device 100 provides for quick installation and removal (<30 seconds per staple). The individual elements 20 may allow for a lower risk of tearing annulus. The discrete elements are less sensitive than sutures to apply and percutaneous applications are feasible.

As discussed in the foregoing, embodiments of the present invention may be adapted for use with a percutaneous technique. The percutaneous technique may leverage existing stearable catheter technologies. Optionally, all actuation may be achieved with simple push-pull motion or existing balloon techniques. The percutaneous device may be off-pump capable. In some embodiments of the present invention, all tool technology can pass through 10 mm percutaneous orifice. The percutaneous may integrate with existing Cribier-Edwards Valve. Optionally, the procedure may be a 30-45 minute, off pump, percutaneous valve replacement. The percutaneous device may also provide flexibility wherein the concepts focus on accommodating wide ranges of sizes, calcification, surgeon proficiency, etc.

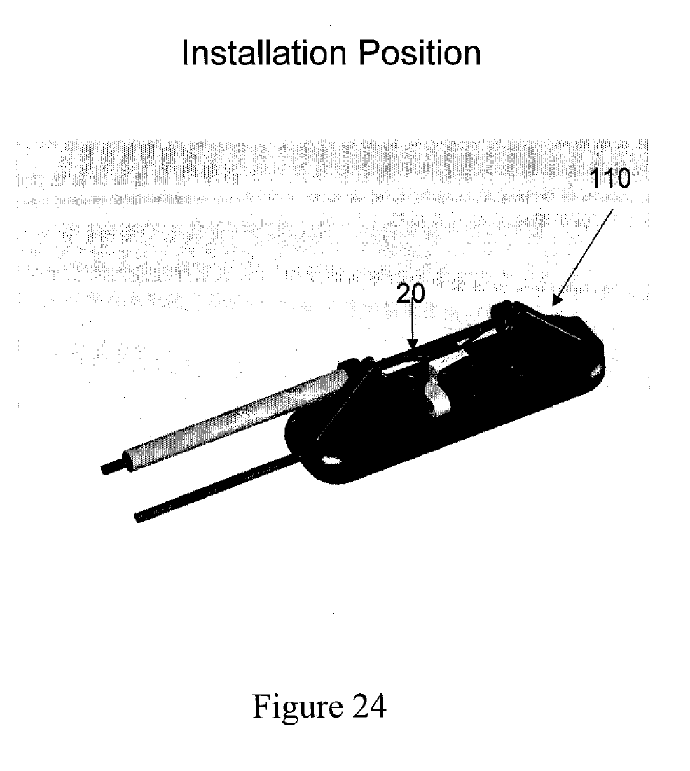

Referring now to FIG. 24, one embodiment of the present invention for use in a percutaneous prosthesis fastening procedure will now be described.

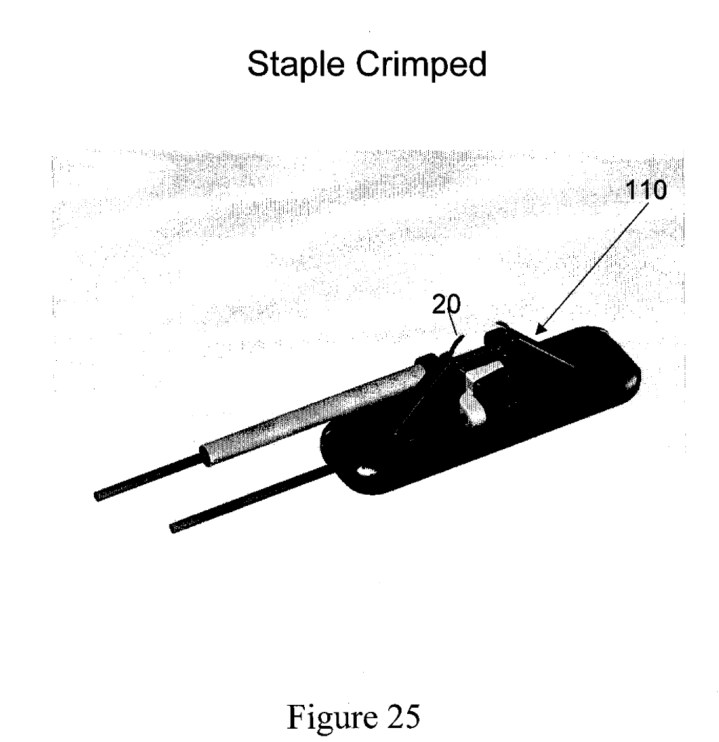

FIG. 24 shows one individual element 20 in a jig 110 that will then be compressed (see FIG. 25) to provide a element 20 in a C-configuration that will grip tissue and a portion of the prosthesis.

FIG. 25 shows the jig 110 compressing the element 20 into a C-configuration. The change into the C-configuration will also crimp the element 20 to engage tissue.

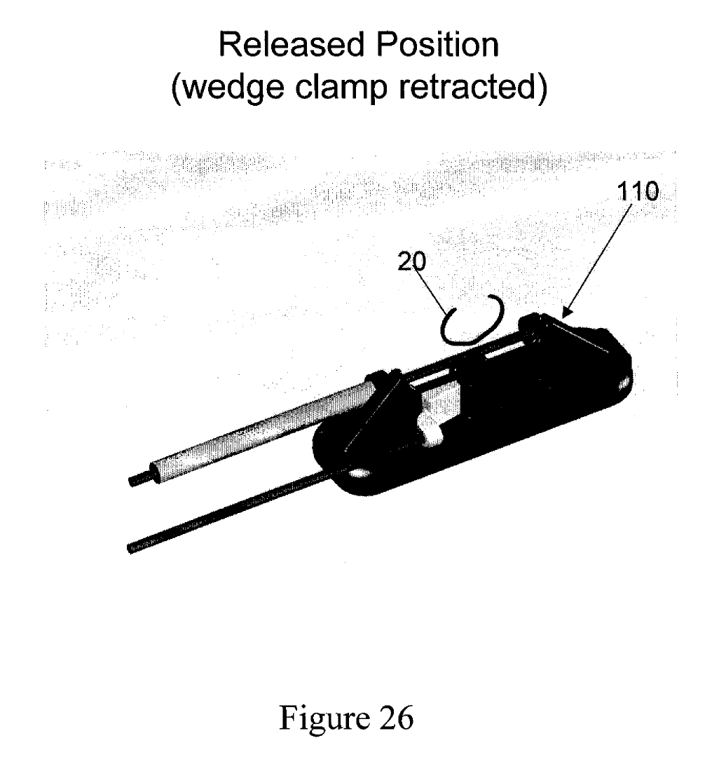

FIG. 26 shows the element 20 being released from the jig 110.

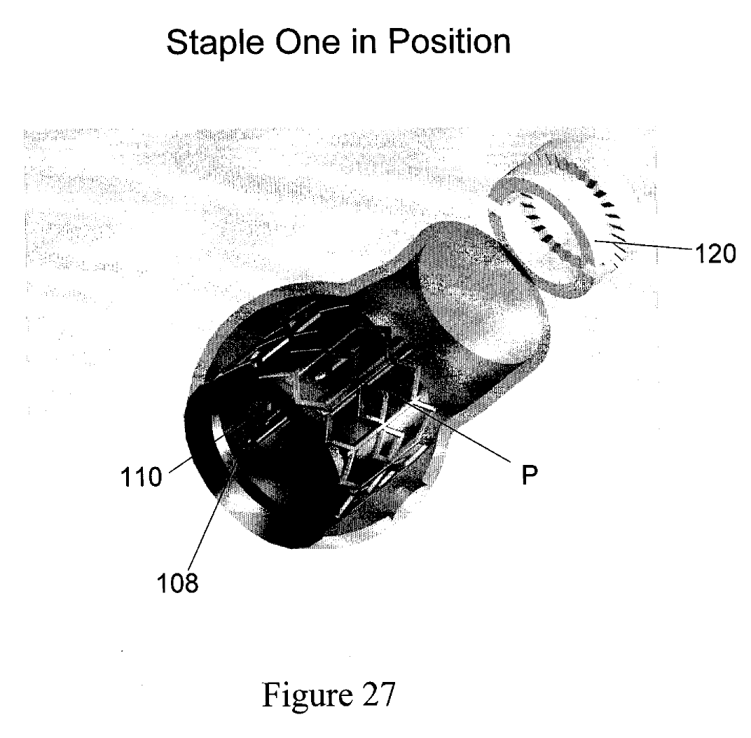

FIG. 27 shows the attachment device 108 with the jig 110 for shaping and delivering the elements 20.

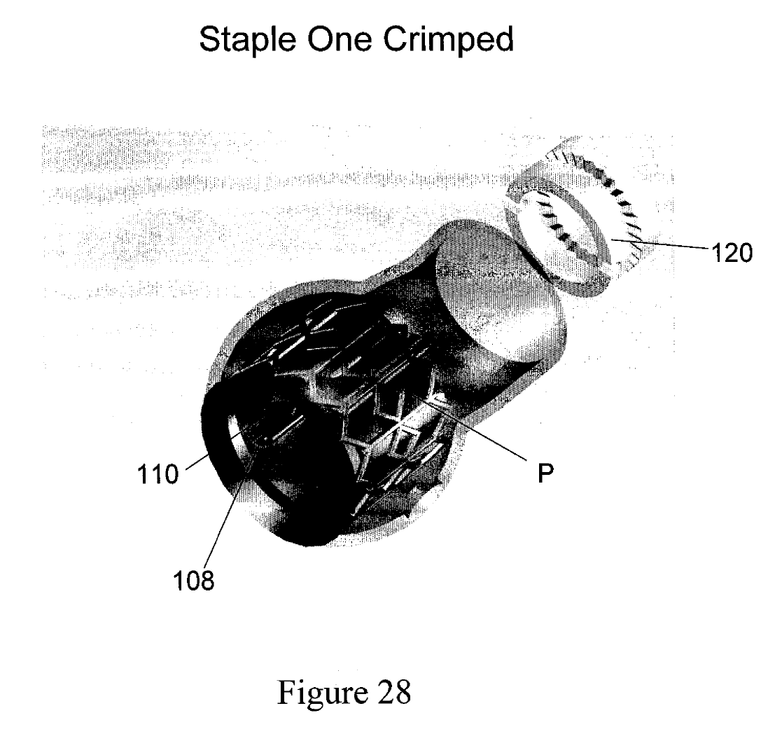

FIG. 28 shows the jig 110 compressing the element 20 into the C-configuration. It should be understood that the device may bend the element 20 into other configurations that can grip tissue and the valve prosthesis.

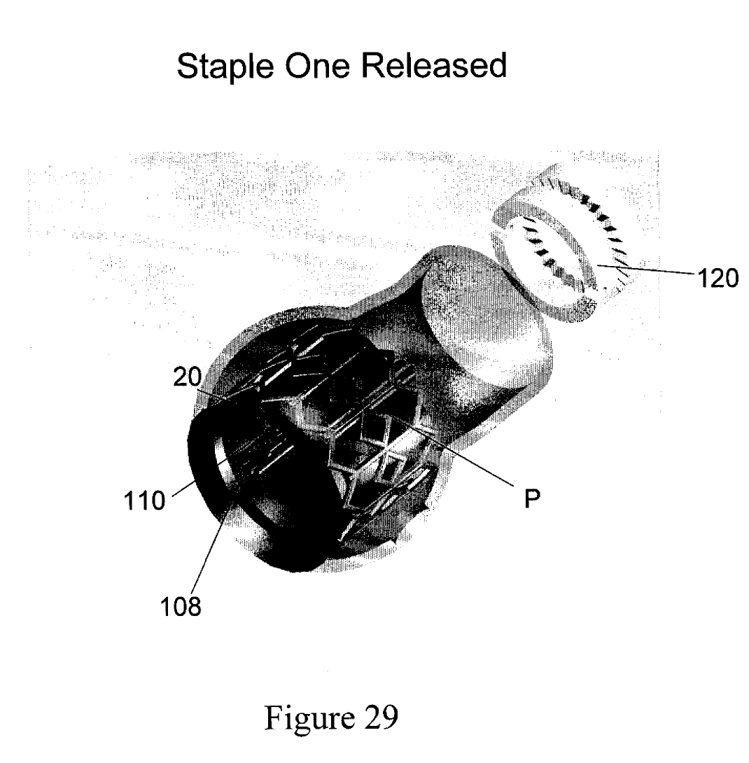

FIG. 29 shows the first element 20 released.

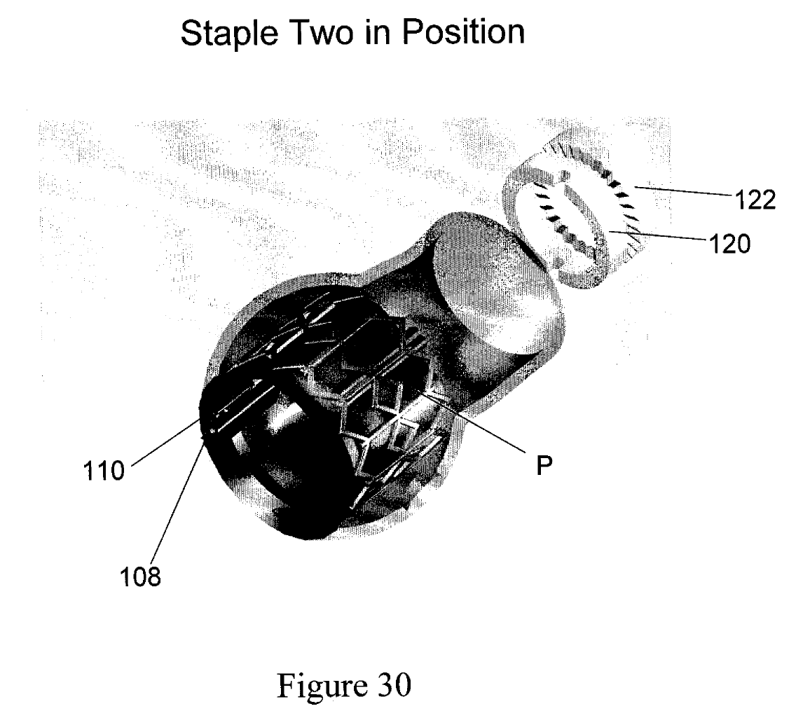

FIG. 30 shows that the rotational device 120 coupled to the geared ring 122 has turned and indexed the device 108 to a different location along the circumference of the valve prosthesis P to deliver an element 20. The device 108 is pressed against the annular portion of the prosthesis P to deliver the element 20.

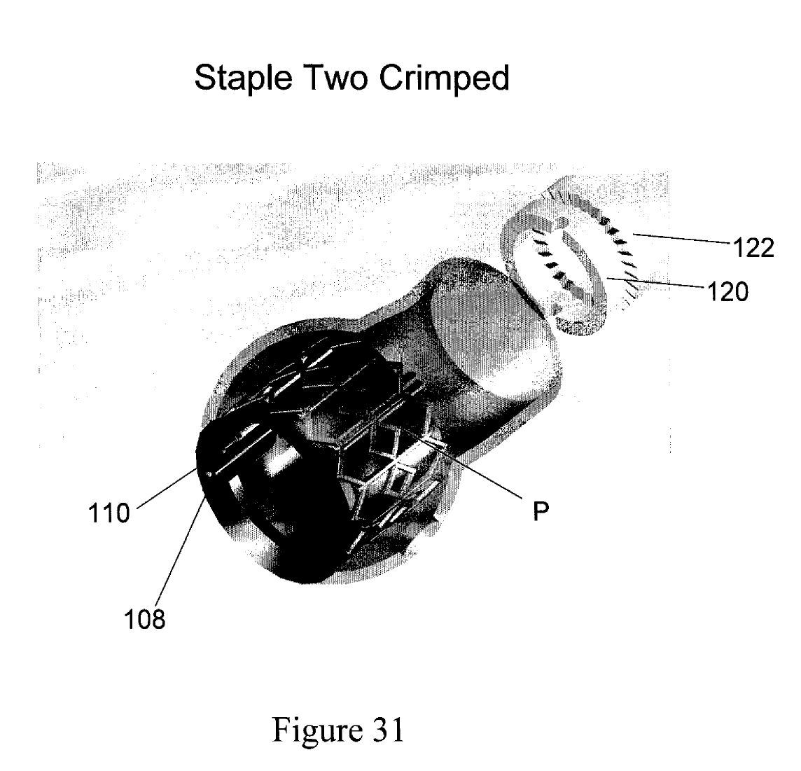

FIG. 31 shows the jig 110 compressing the element 20 into the C-configuration. It should be understood that the device may bend the element 20 into other configurations that can grip tissue and the valve prosthesis.

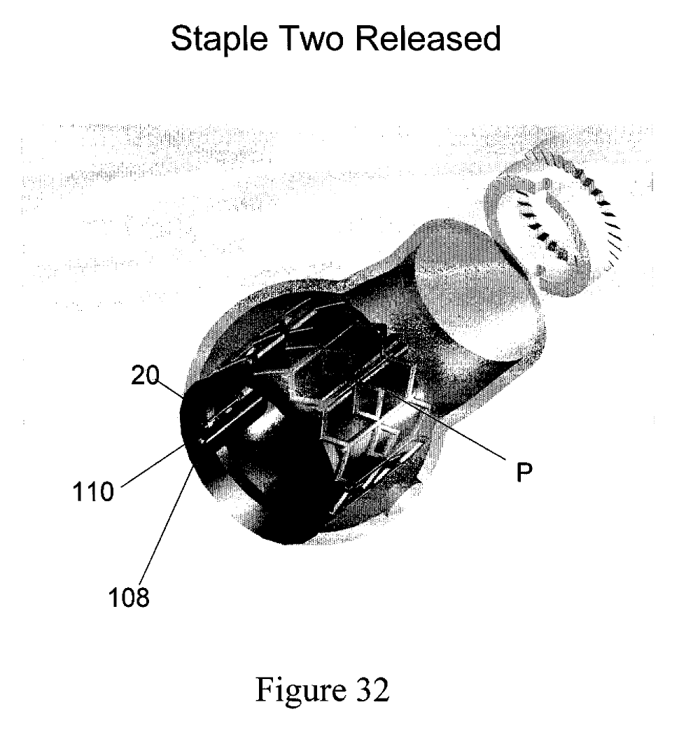

FIG. 32 shows the second element 20 released.

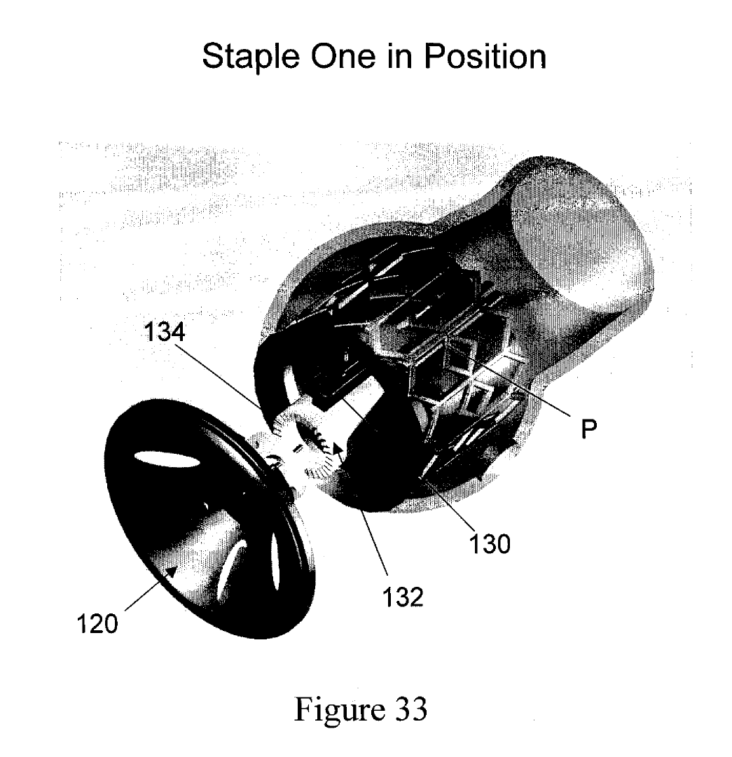

Referring now to FIG. 33, another embodiment of the present invention will now be described. The embodiment of this figure shows a temporary valve 120 and a fastening device 130 mounted about a central shaft 132 which will rotate to index the fastening device 130 along the circumference of the valve prosthesis P to attach the discrete elements 20. As seen, the shaft 132 may have a geared portion 134 to facilitate the rotation to index the device 130.

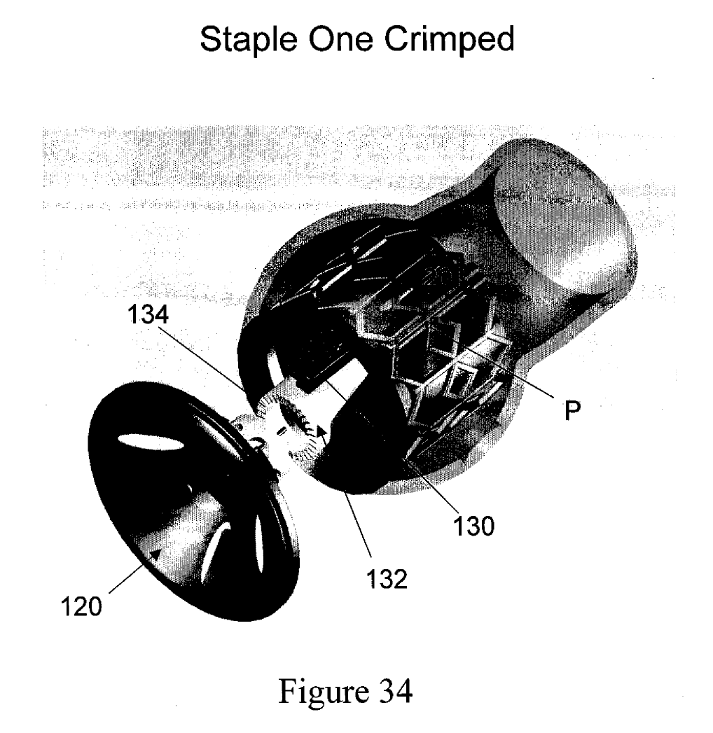

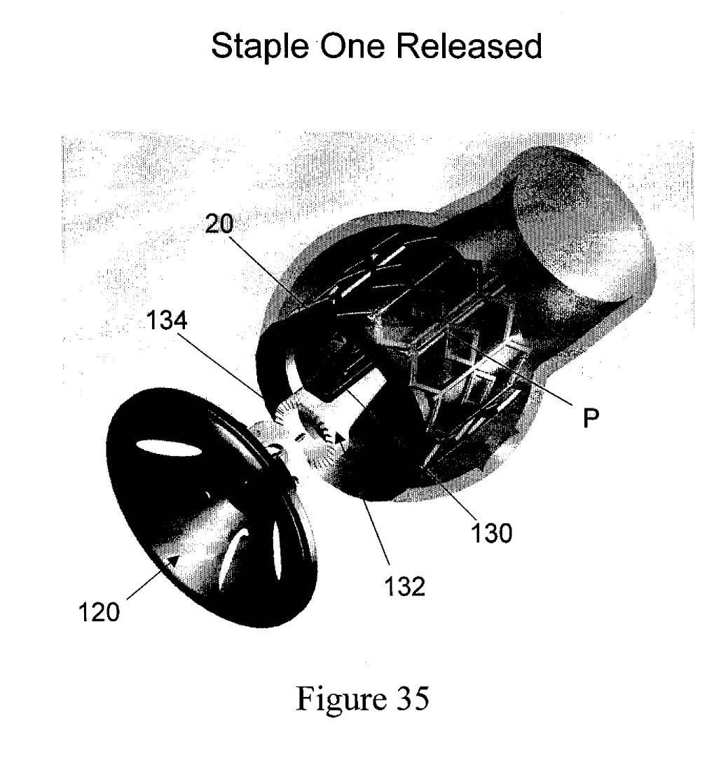

FIGS. 34 through 35 show the crimping and releasing of the fastener 20.

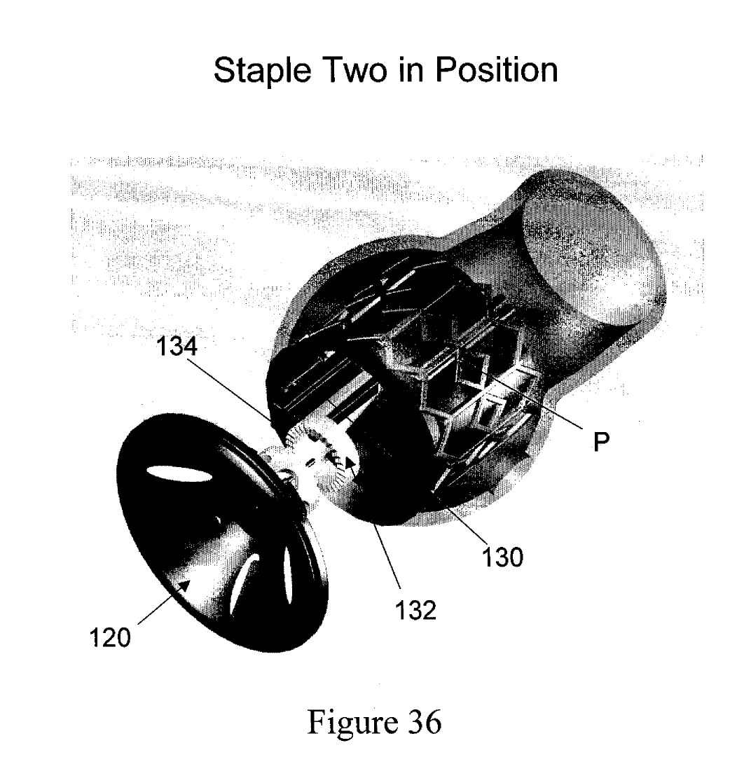

FIG. 36 shows that the shaft 132 may be rotated to reposition the device 130 to deliver and/or crimp the elements 20 in various locations.

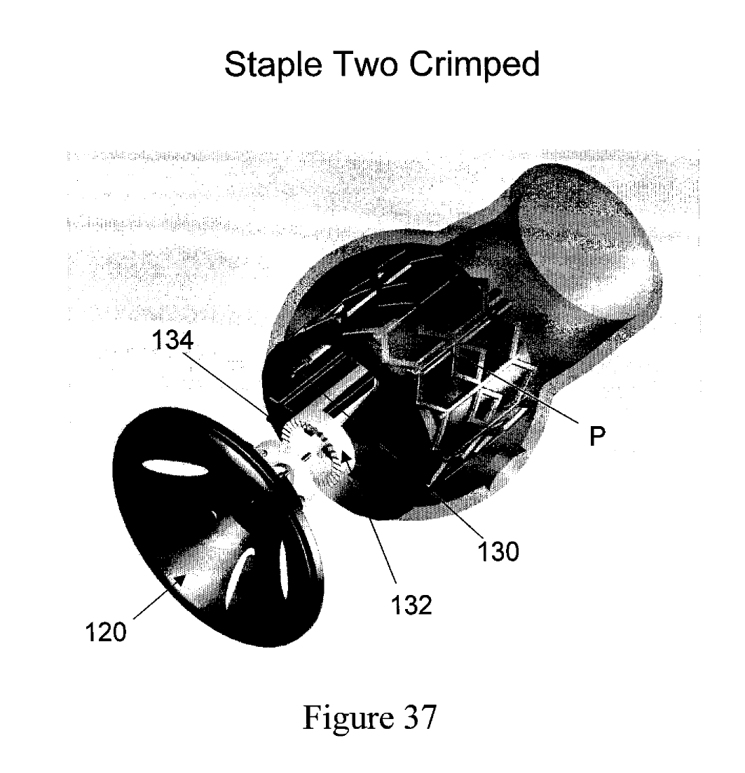

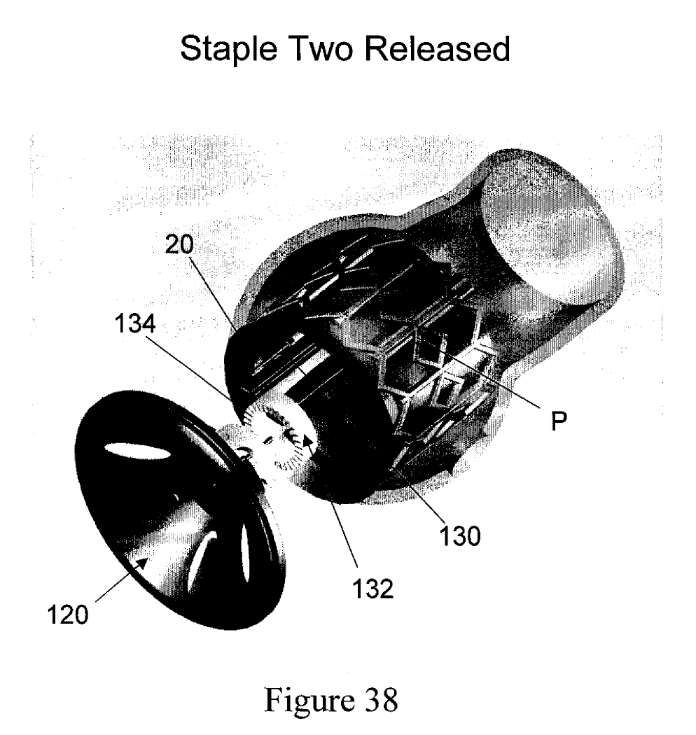

FIGS. 37 through 38 show the crimping and releasing of the fastener 20 at a second location.

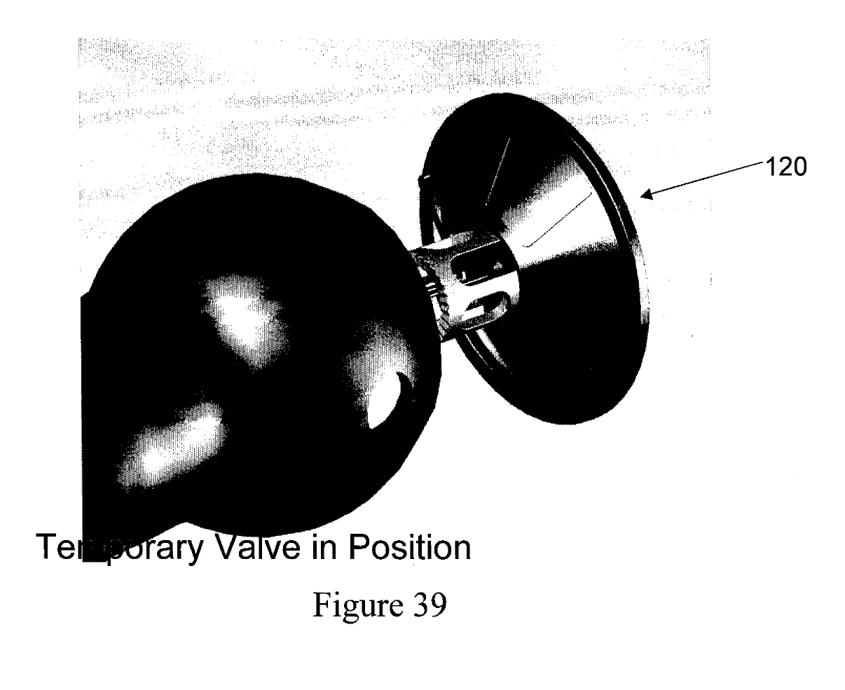

FIG. 39 shows another view of the temporary valve 120.

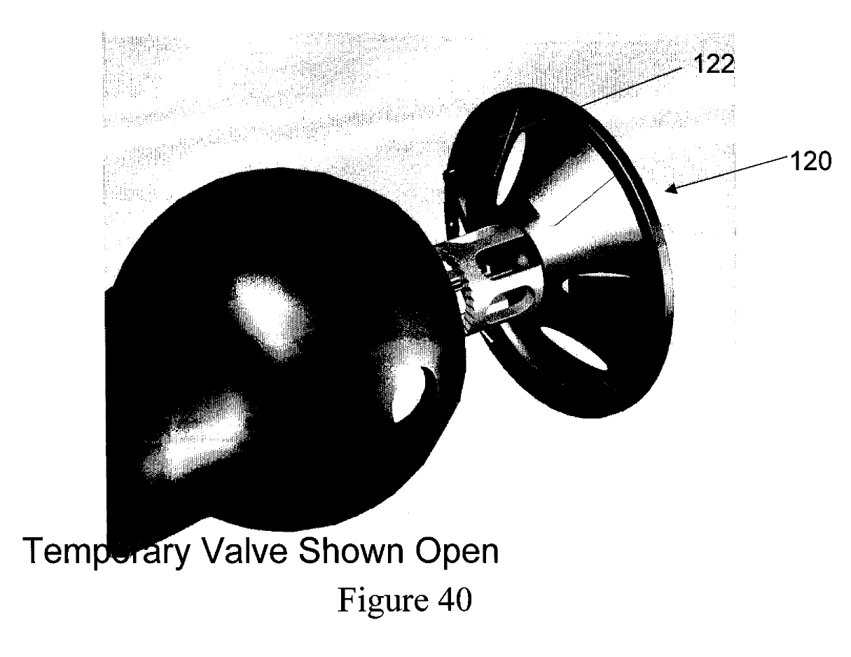

FIG. 40 shows the temporary valve 120 in an open configuration with the flaps 122 opened.

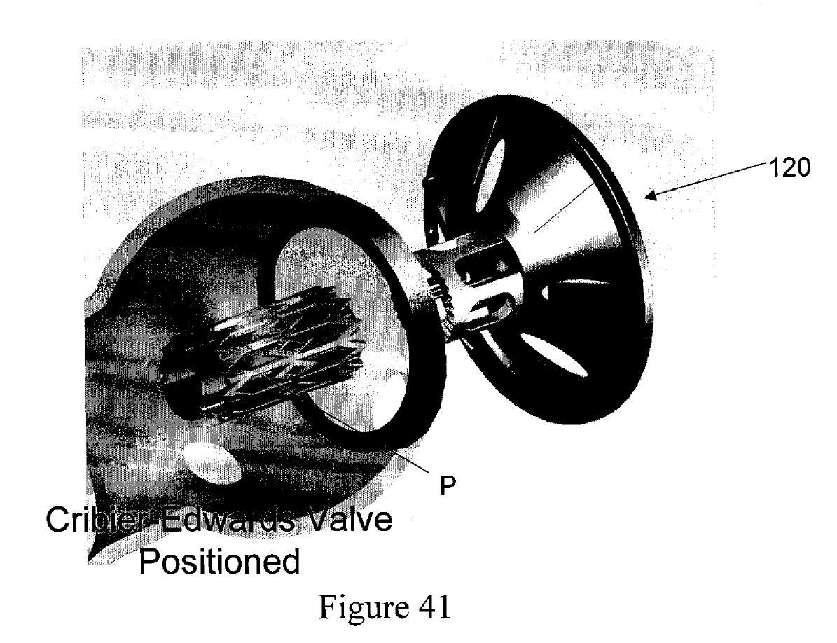

FIG. 41 shows a cross-sectional view more clearly showing a compressed Cribier-Edwards Valve positioned for deployment.

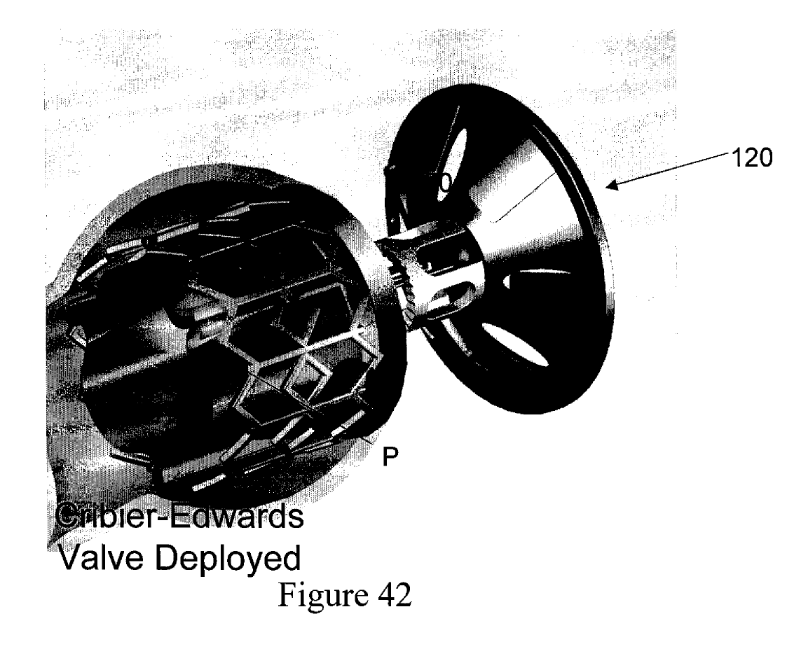

FIG. 42 shows the Cribier-Edwards Valve deployed in place and ready for attachment.

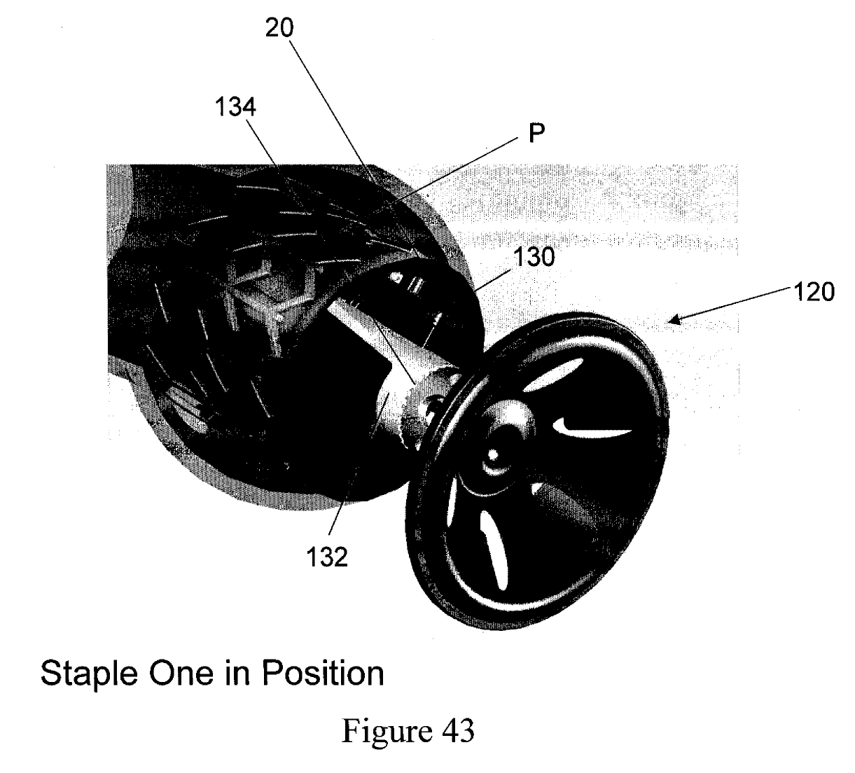

FIG. 43 shows the device 130 ready to deploy and crimp the element 20.

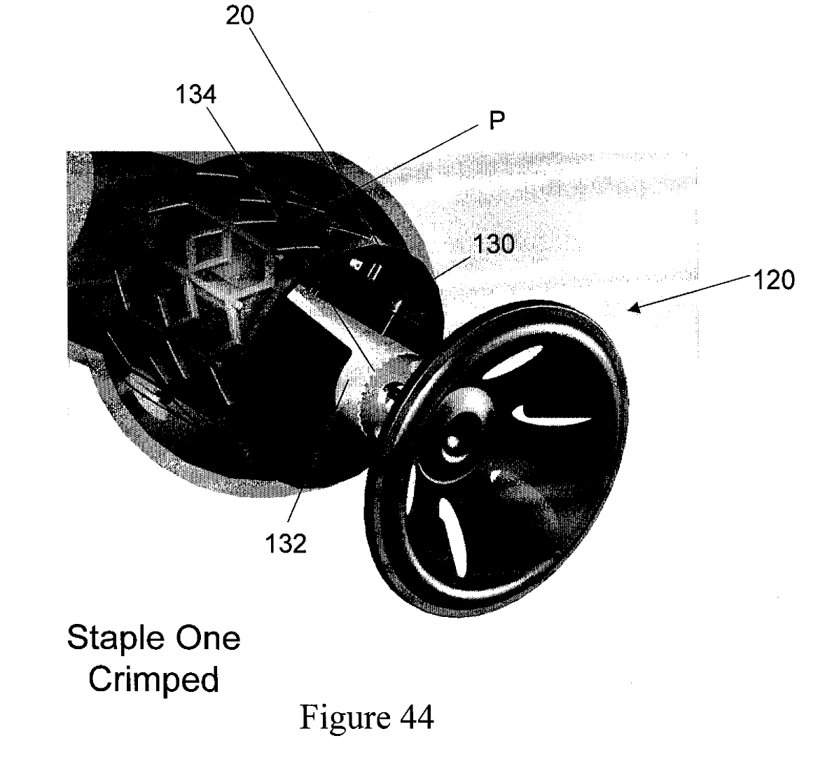

FIG. 44 shows the element 20 being crimped.

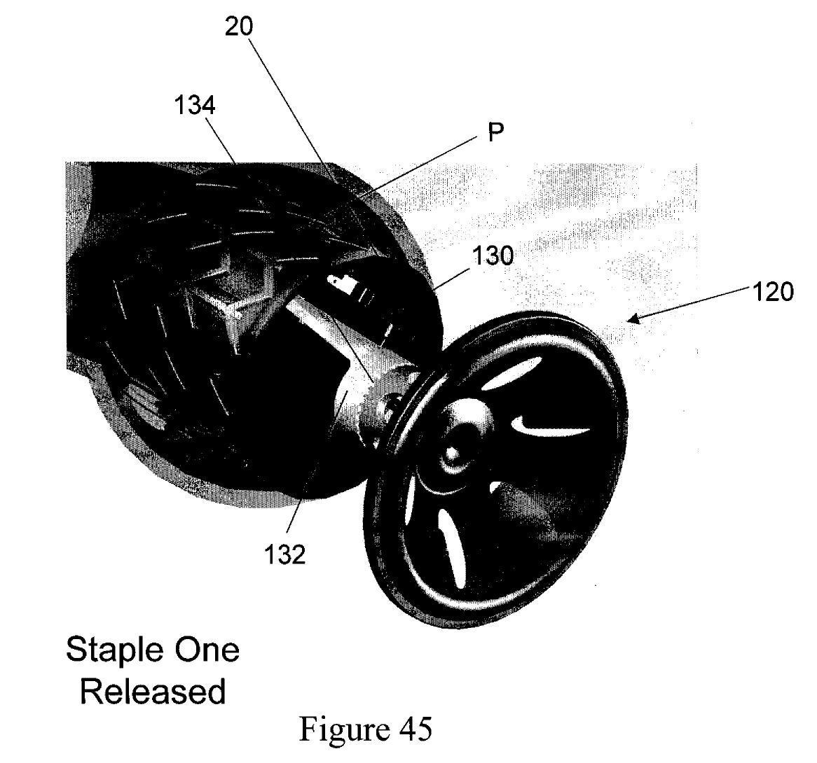

FIG. 45 shows the device 130 lowering the jig 110 away from the element 20 to release it.

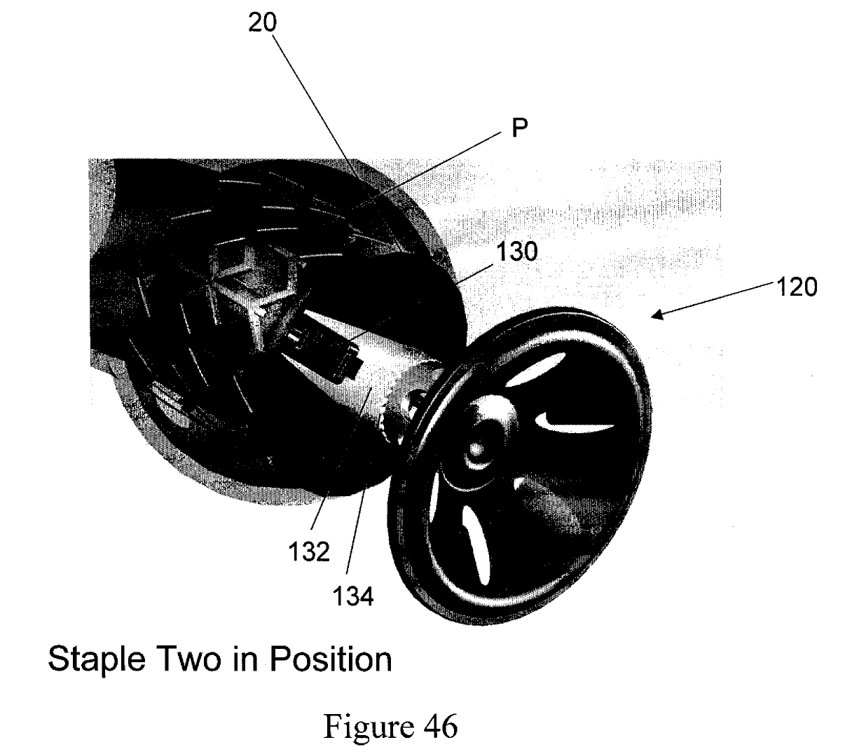

FIG. 46 shows the rotation of the shaft 132 to position the device 130 in a new location to deploy another fastener 20.

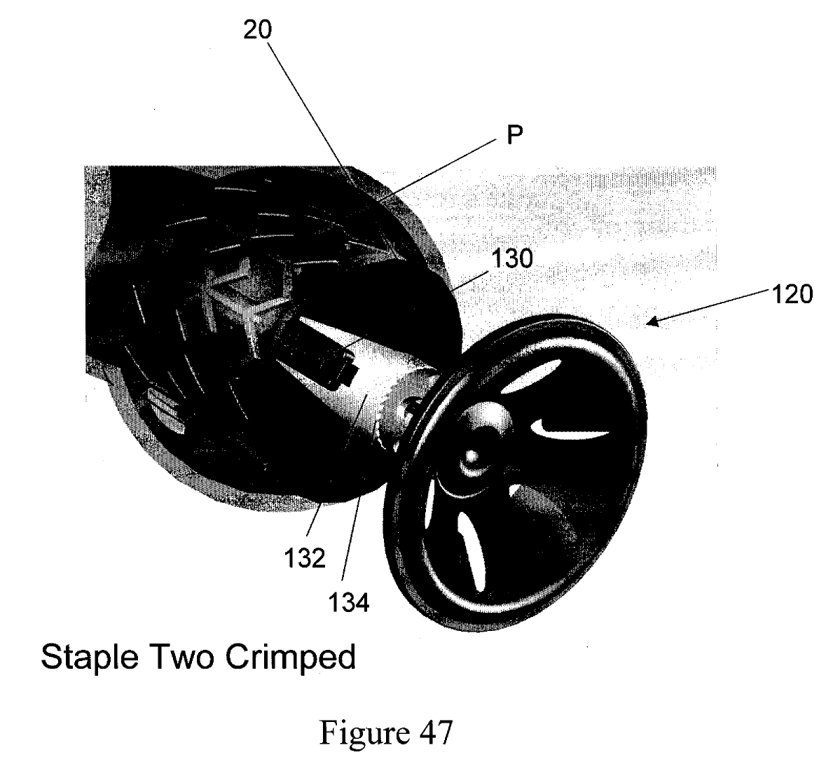

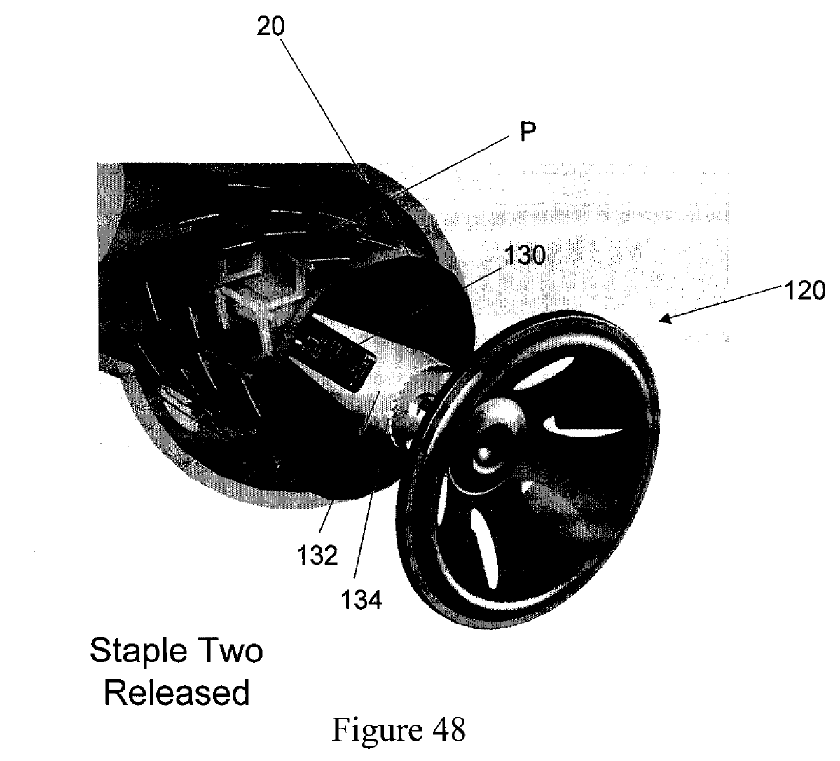

FIGS. 47 and 48 show the crimp and release of the fastener 20.

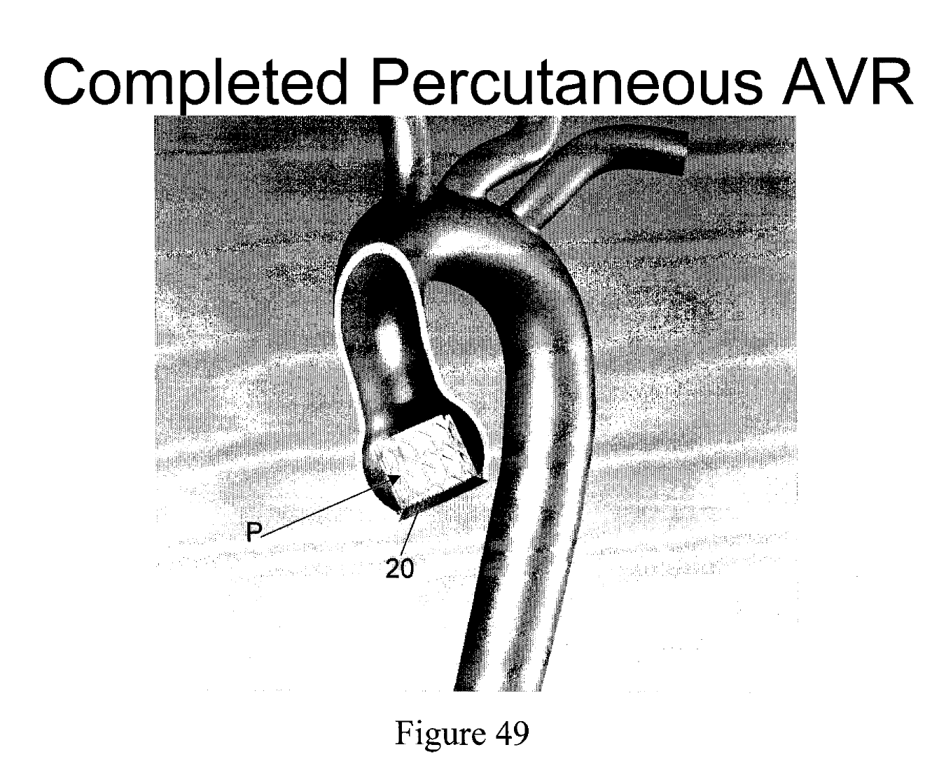

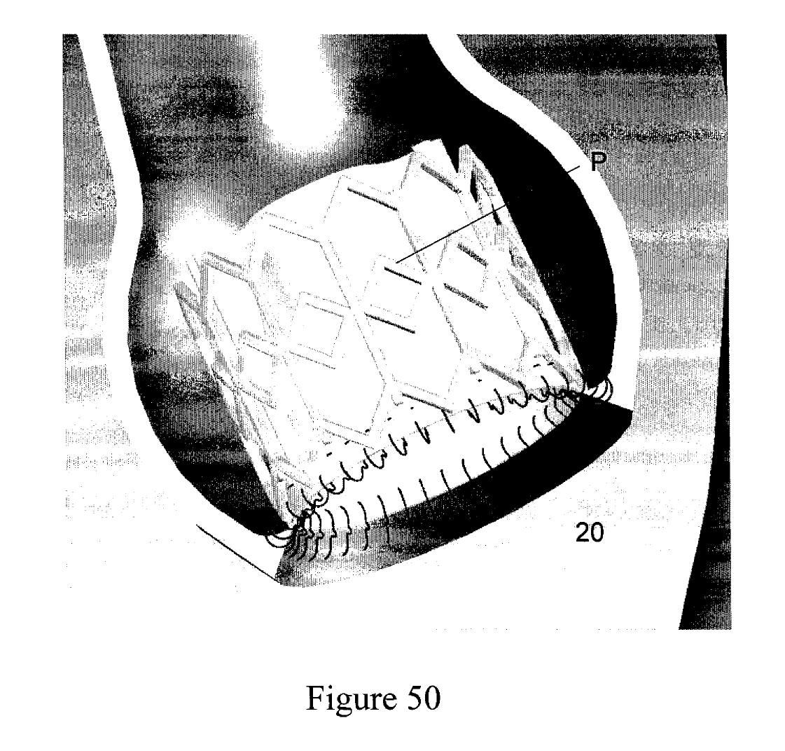

FIGS. 49 and 50 show cross-sectional view of the prosthesis P in place and attached by a plurality of discrete elements 20.

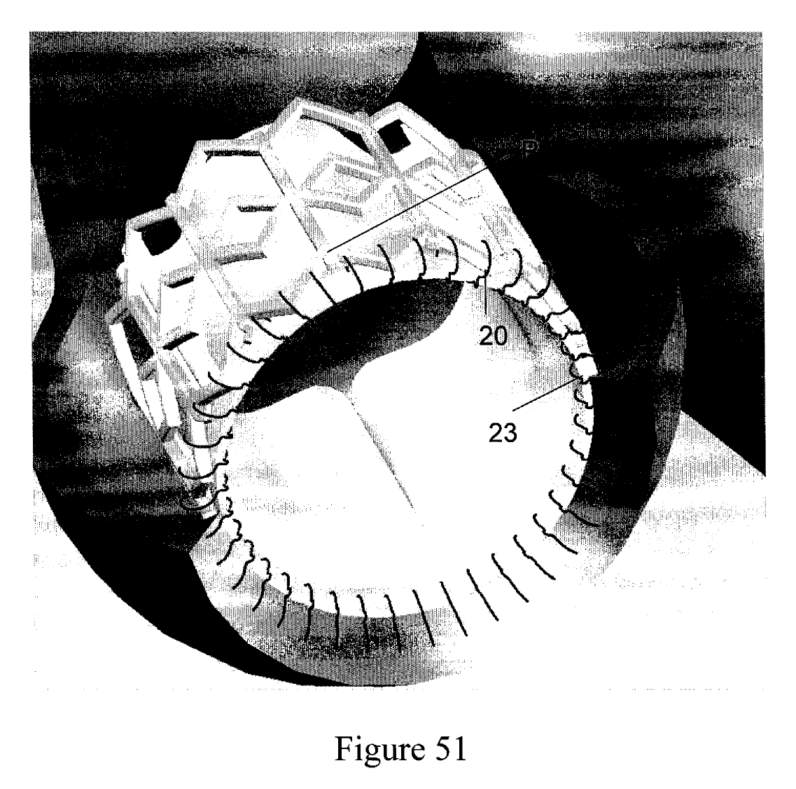

As seen in FIG. 51, the shape of a crimped element 20 may include a rise or bump 23 along a mid-section to grip the prosthesis P. The crimped shape, however, may be varied as desired and may be U-shaped, M-shaped, or combination of shapes.

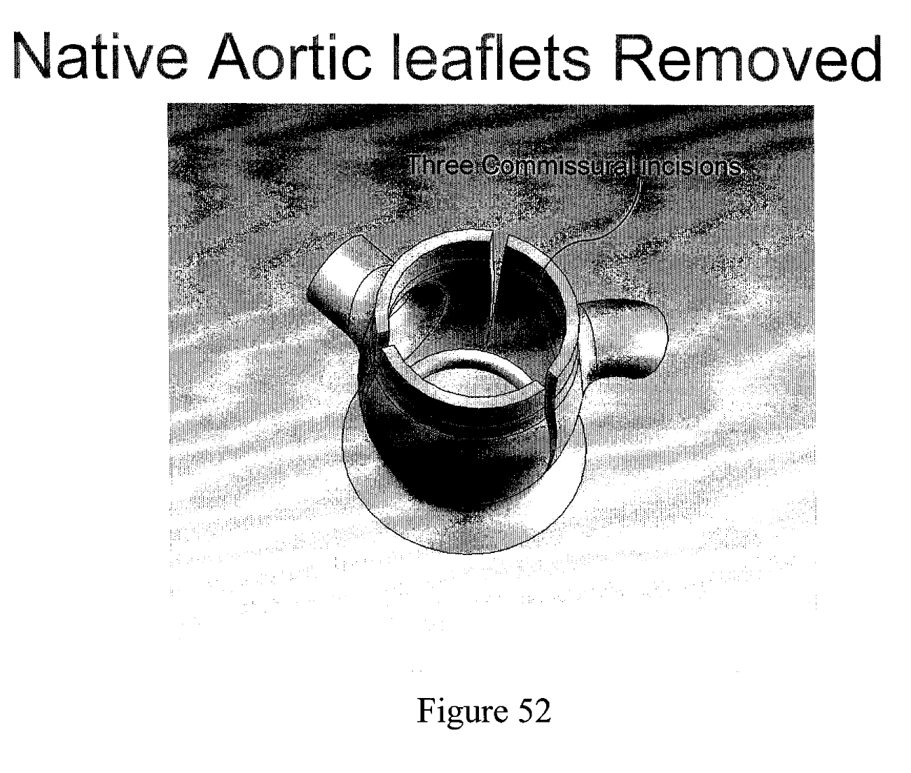

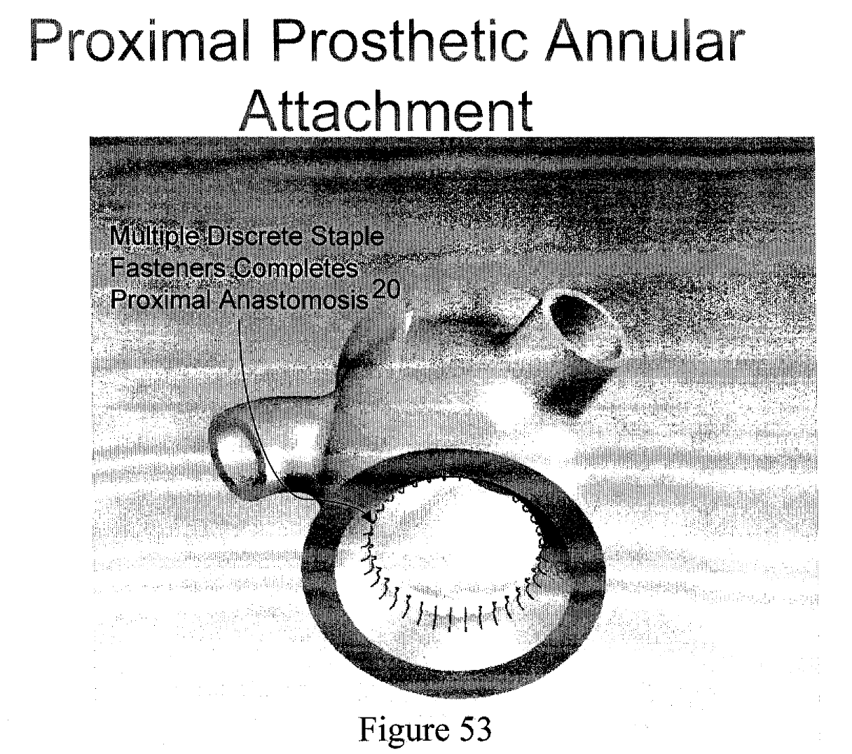

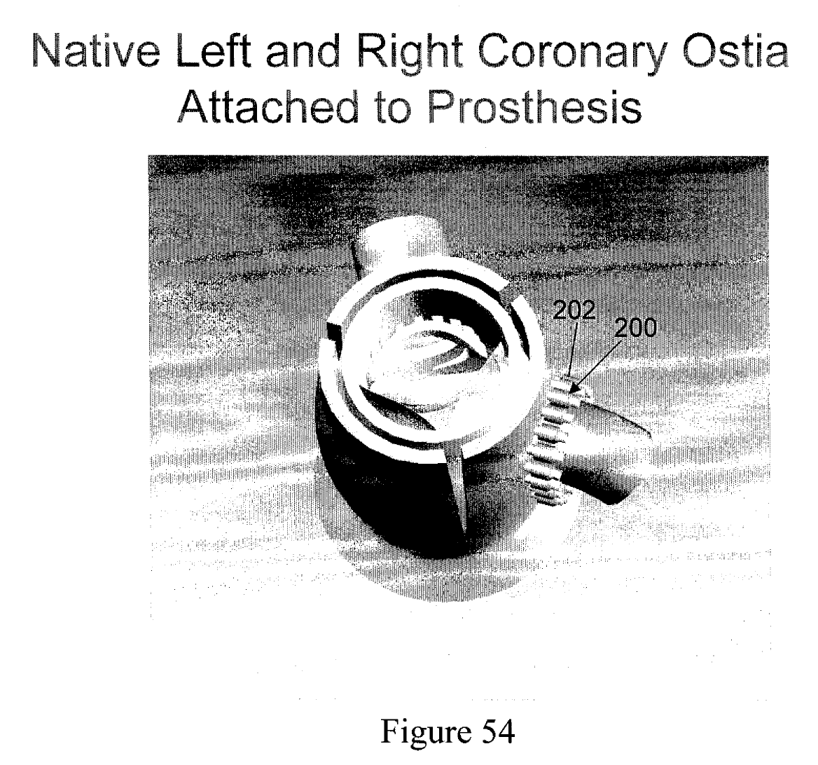

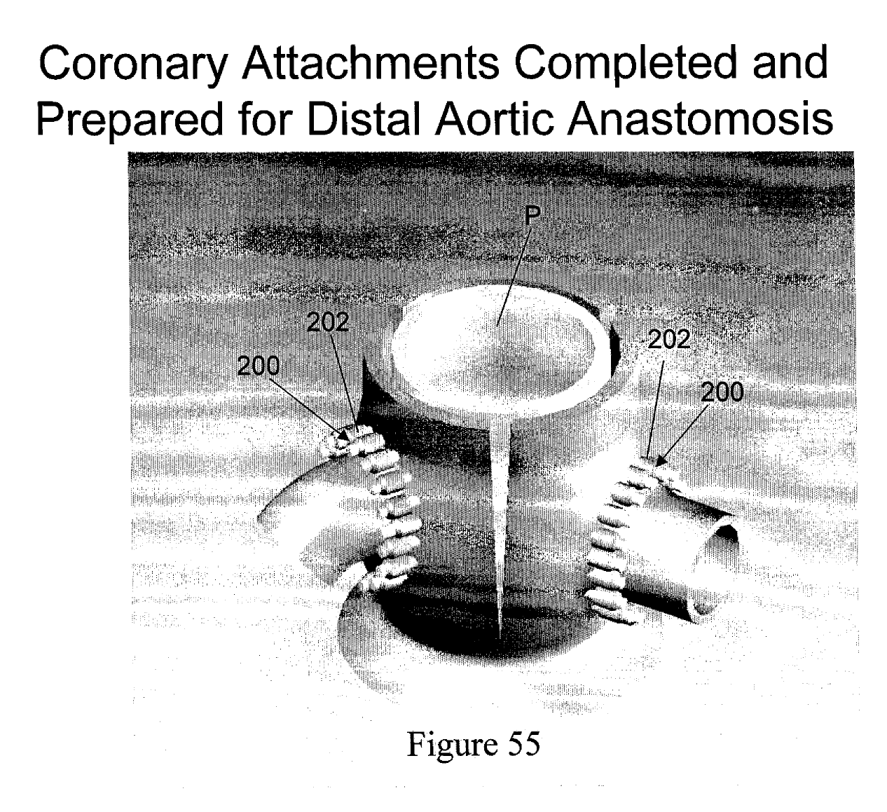

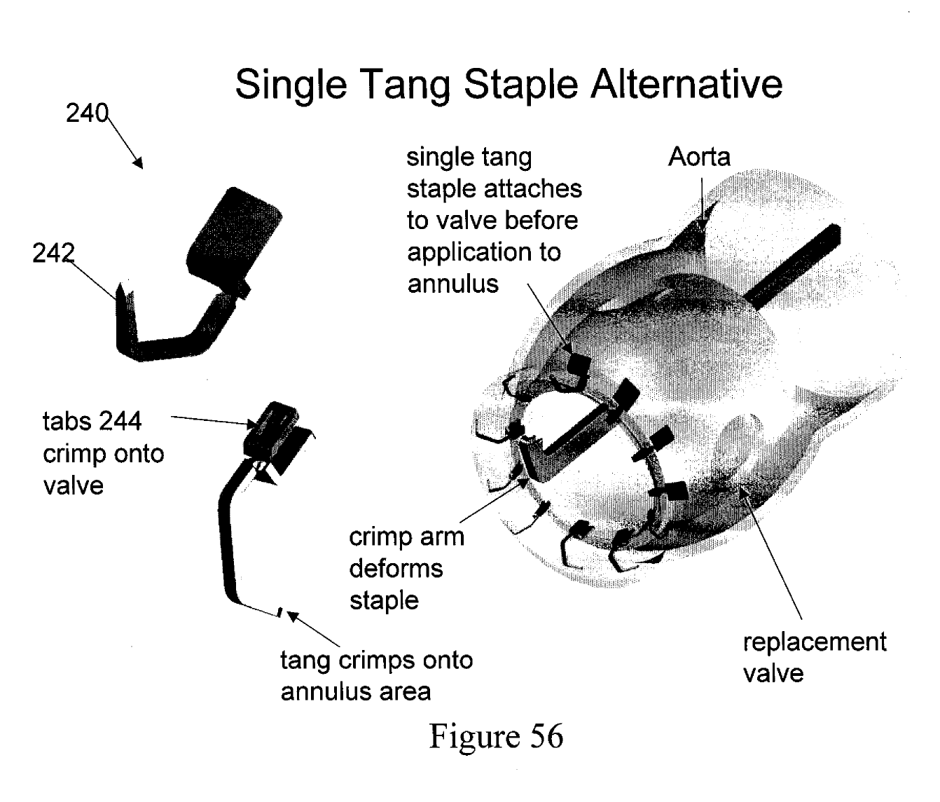

FIGS. 52 through 55 show aortic root attachment methods. The prosthesis may be attached by a plurality of discrete elements 20. The attachment of the prosthesis to the root near the native left and right coronary ostia may use fasteners 200 which piece through the prosthesis and vessel wall. They are held in place by barb like structures inside the collar 202. FIG. 55 shows coronary attachments completed and prepared for distal aortic anastomosis. FIG. 56 shows an single tang staple alternative.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, with any of the above embodiments, a prosthetic valve or a graft may be premounted on to the apparatus. With any of the above embodiments, the apparatus may be configured to be delivered percutaneously or through open surgery. With any of the embodiments herein, the devices may be attached by a variety of techniques including sutures, preattached sutures and needles, shape memory clips that will engage tissue, anchors, other fastener device, or any combination of the above. It should be understood that the present invention may be adapted for use on other valves throughout the body. Embodiments of the present invention may be used with stented, stentless, mechanical, or other valves. Some embodiments may be used in open surgery or for off-pump, minimally invasive techniques. These catheters may have sheaths that retract to reveal the active portions of the anvil and the cutter to allow for deployment. The catheter may be coaxially mounted about the guidewire or in some embodiments, they may have extensions or arms that follow the guidewire while the catheter itself is spaced apart from the guidewire. With any of the embodiments, there may be alterative embodiments with only a tent and no embolic screen and vice versa. With any of the above embodiments, there may be more than one tent or more than one embodiment screen. Some embodiments may have two, three, or four embolic screens. Some may have embolic screens made of more than one piece. With any of the embodiments, it should be understood that the embolic screen and tent may be used with cutters of other configurations and valve fasteners of other configurations than those shown herein.

In other embodiments, the number of fasteners per prosthesis can vary. Some prosthesis may have 2, 3, 4, 5, 6, 7, 8, 9, 10, 11, 12, 13, 14, 15, 20, 25, 30, 35, 40, or more individual barbs, fasteners, hooks or the like. Some embodiments may have different types or shapes to their fasteners. Some may have hooks or barbs of varying length. Some embodiments may also have four balloons and four bands. Embodiments may also include fasteners to pincer tissue.

The publications discussed or cited herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All publications mentioned herein are incorporated herein by reference to disclose and describe the structures and/or methods in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A percutaneous valve replacement assembly comprising:
a catheter;
a plurality of discrete elements for tissue attachment; and
a fastener device mounted to the catheter;
wherein the fastener device includes a working end that is movable to a first configuration to engage one of the discrete elements and a second configuration to crimp the discrete element into target tissue.

2. The valve replacement assembly of claim 1 further comprising:
a debris tent positioned over the valve cutter to capture debris created by the valve cutter during tissue removal.

3. The valve replacement assembly of claim 1 further comprising:
a expandable temporary valve coupled to the catheter to regulate blood flow during delivery of the expandable valve prosthesis.

4. The valve replacement assembly of claim 1 further comprising:
a shaft having a geared portion to rotate the fastening device to different radial positions to engage different tissue areas.

5. The device of claim 2 further comprising an embolic screen positioned downstream from the debris tent.

6. The device of claim 2 wherein the valve cutter, debris tent, and embolic screen are all positioned over a catheter.

7. The device of claim 1 further comprising a valve prosthesis mounted on a catheter coupled to the valve cutter and the debris tent.

8. A method of percutaneous valve replacement, the method comprising:
accessing a femoral blood vessel and inserting a guidewire to guide a catheter to a target site in the heart;
advancing the catheter along the guidewire in a collapsed configuration;
positioning at least one of a plurality of discrete elements on the catheter for tissue attachment;
using a fastener device mounted to the catheter, wherein the fastener device includes a working end that is movable to a first configuration to engage one of the discrete elements and a second configuration to crimp the discrete element into target tissue.

9. The method of claim 8 wherein the prosthesis and the discrete elements are on separate catheters.

10. The method of claim 8 wherein the prosthesis and the discrete elements are on the same catheter.

11. The method of claim 8 further comprising an embolic screen that is deployed downstream from the target site to capture debris from the valve.

12. The method of claim 8 wherein prosthesis includes a stent.

13. The method of claim 8 further comprising pushing a plunger to crimp the discrete elements to couple to tissue.

14. The method of claim 8 further comprising rotating a shaft to index the fastening device to a new location.

15. The method of claim 8 further comprising attaching said valve apparatus at the ventriculo-arterial junction.

16. The method of claim 8 further comprising driving said penetrating members through the valve prosthesis to anchor the prosthesis to the target tissue.

17. A minimally invasive prosthesis attachment device comprising:
a shaft;
a plurality of discrete elements for tissue attachment;
a clamp system for holding each of the discrete elements in position to engage target tissue; and
a fastener device mounted to the shaft;
wherein the fastener device includes a working end that is movable to a first configuration to engage one of the discrete elements and a second configuration to crimp the discrete element into target tissue..

18. The device of claim 17 wherein the shaft has an elongate portion, a distal end and a proximal end.

19. The device of claim 17 further comprising a plunger longitudinally slidable to crimp each element and advance the element into target tissue.

20. The device of claim 17 further comprising a pericardial tent positioned to capture valve leaflets between the tent and the valve excisor.

21. The device of claim 17 further comprising an embolic screen mounted on said second apparatus.

22. The device of claim 17 further comprising a pericardial tent on said second apparatus and formed of a mesh and positioned to capture valve leaflets between the tent and the valve excisor.

23. The device of claim 17 wherein said penetrating members are made of nitinol.

24. The device of claim 17 wherein said penetrating members are made of stainless steel.
